(19) United States
(12) Patent Application Publication
(10) Pub. No.: US 2011/0286969 A1
(43) Pub. Date: Nov. 24, 2011

(54) DEUTERATED COMPOUNDS AS HEPATITIS C VIRUS (HCV) INHIBITORS

(75) Inventors: Malcolm MacCoss, Seabrook Island, SC (US); F. George Njoroge, Warren, NJ (US); Frank Bennett, Cranford, NJ (US); Srikanth Venkatraman, Edison, NJ (US); Joseph A. Kozlowski, Princeton, NJ (US); Wensheng Yu, Edison, NJ (US); Paul E. McNamara, Scotch Plains, NJ (US); Pernilla R. Royster, Roselle Park, NJ (US)

(73) Assignee: SCHERING CORPORATION, KENILWORTH, NJ (US)

(21) Appl. No.: 13/139,131

(22) PCT Filed: Dec. 10, 2009
(86) PCT No.: PCT/US09/67415

§ 371 Date: Aug. 10, 2011

(30) Foreign Application Priority Data
Dec. 12, 2008 (US) 61/122083

(51) Int. Cl. A61K 38/21; A61P 31/14; C12N 9/99; C07K 5/103; A61K 38/07

(52) U.S. Cl. 424/85.7; 530/330; 514/4.3; 424/85.4; 435/184

(57) ABSTRACT

The present invention discloses novel compounds which have HCV protease inhibitory activity as well as methods for preparing such compounds In another embodiment, the invention discloses pharmaceutical compositions comprising such compounds as wet! as methods of using them to treat disorders associated with the HCV protease An illustrative inventsve compound is shown below: Formula (I).

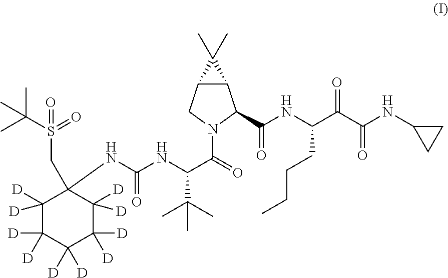

### FIELD OF THE INVENTION

The present invention relates to novel deuterated compounds as hepatitis C virus (“HCV”) protease inhibitors, pharmaceutical compositions containing one or more such compounds, methods of preparing such compounds and methods of using such inhibitors to treat hepatitis C and related disorders. This invention additionally discloses novel compounds as inhibitors of the HCV NS3/NS4a serine protease.

### BACKGROUND OF THE INVENTION

Hepatitis C virus (HCV) is a (+)-sense single-stranded RNA virus that has been implicated as the major causative agent in non-A, non-B hepatitis (NANBH), particularly in blood-associated NANBH (BB-NANBH) (see, International Patent Application Publication No. WO 89/04669 and European Patent Application Publication No. EP 381 216). NANBH is to be distinguished from other types of viral-induced liver disease, such as hepatitis A virus (HAV), hepatitis B virus (HBV), delta hepatitis virus (HDV), cytomegalovirus (CMV) and Epstein-Barr virus (EBV), as well as from other forms of liver disease such as alcoholism and primary biliar cirrhosis.

Recently, an HCV protease necessary for polypeptide processing and viral replication has been identified, cloned and expressed. (See, e.g., U.S. Pat. No. 5,712,145). This approximately 3000 amino acid polyprotein contains, from the amino terminus to the carboxy terminus, a nucleocapsid protein (C), envelope proteins (E1 and E2) and several non-structural proteins (NS1, 2, 3, 4a, 5a and 5b). NS3 is an approximately 68 kda protein, encoded by approximately 1893 nucleotides of the HCV genome, and has two distinct domains: (a) a serine protease domain consisting of approximately 200 of the N-terminal amino acids; and (b) an RNA-dependent ATPase domain at the C-terminus of the protein. The NS3 protease is considered a member of the chymotrypsin family because of similarities in protein sequence, overall three-dimensional structure and mechanism of catalysis. Other chymotrypsin-like enzymes are elastase, factor Xa, thrombin, trypsin, plasmin, urokinase, tPA and PSA. The HCV NS3 serine protease is responsible for proteolysis of the polypeptide (polyprotein) at the NS3/NS4a, NS4a/NS4b, NS4b/NS5a and NS5a/NS5b junctions and is thus responsible for generating four viral proteins during viral replication. This has made the HCV NS3 serine protease an attractive target for antiviral chemotherapy. The inventive compounds can inhibit such protease. They also can modulate the processing of hepatitis C virus (HCV) polypeptide.

It has been determined that the NS4a protein, an approximately 6 kda polypeptide, is a co-factor for the serine protease activity of NS3. Autocleavage of the NS3/NS4a junction by the NS3/NS4a serine protease occurs intramolecularly (i.e., cis) while the other cleavage sites are processed intermolecularly (i.e. trans).

Analysis of the natural cleavage sites for HCV protease revealed the presence of cysteine at P1 and serine at P1′ and that these residues are strictly conserved in the NS4a/NS4b, NS4b/NS5a and NS5a/NS5b junctions. The NS3/NS4a junction contains a threonine at P1 and a serine at P1′. The Cys→Thr substitution at NS3/NS4a is postulated to account for the requirement of cis rather than trans processing at this junction. See, e.g., Pizzi et al. (1994) Proc. Natl. Acad. Sci. (USA) 91:888-892, Failla et al. (1996) Folding & Design 1:35-42. The NS3/NS4a cleavage site is also more tolerant of mutagenesis than the other sites. See, e.g., Kollykhalov et al. (1994) J. Virol. 68:7525-7533. It has also been found that acidic residues in the region upstream of the cleavage site are required for efficient cleavage. See, e.g., Komoda et al. (1994) J. Virol. 68:7351-7357.

Inhibitors of HCV protease have been reported. See, for example, commonly-owned US 2007/0042968 published Feb. 22, 2007, and references cited therein. The disclosures of US 2007/0042968 and references cited therein are incorporated herein in their entirety by reference thereto.

There is a need for new treatments and therapies for HCV infection.

There is a need for compounds useful in the treatment or prevention or amelioration of one or more symptoms of hepatitis C.

There is a need for methods of treatment or prevention or amelioration of one or more symptoms of hepatitis C.

There is a need for methods for modulating the activity of serine proteases, particularly the HCV NS3/NS4a serine protease, using the compounds provided herein.

There is a need for methods of modulating the processing of the HCV polypeptide using the compounds provided herein.

### DESCRIPTION OF THE INVENTION

Among its many embodiments, US 2007/0042968 published Feb. 22, 2007, discloses a compound of the general formula:

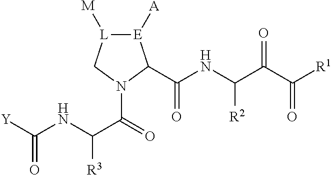

where the various moieties are defined therein. A particularly interesting compound disclosed therein as a HCV protease inhibitor has formula I:

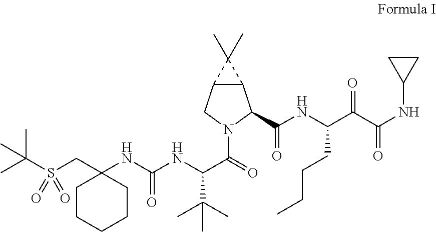

In its many embodiments, the present invention provides certain novel deuterated compounds (Formulas II-XXXVIXIV), as well as pharmaceutically acceptable salts, solvates or esters of said compounds, as inhibitors of the HCV protease, pharmaceutical compositions containing one or more of the compounds, methods of preparing pharmaceutical formulations comprising one or more such compounds, and methods of treatment or prevention of HCV or amelioration of one or more of the symptoms of hepatitis C using one or more such compounds or one or more such formulations. Also provided are methods of modulating the interaction of an HCV polypeptide with HCV protease. Among the compounds provided herein, compounds that inhibit HCV NS3/NS4a serine protease acV are the deuterated analogs of the compound of Formula I.

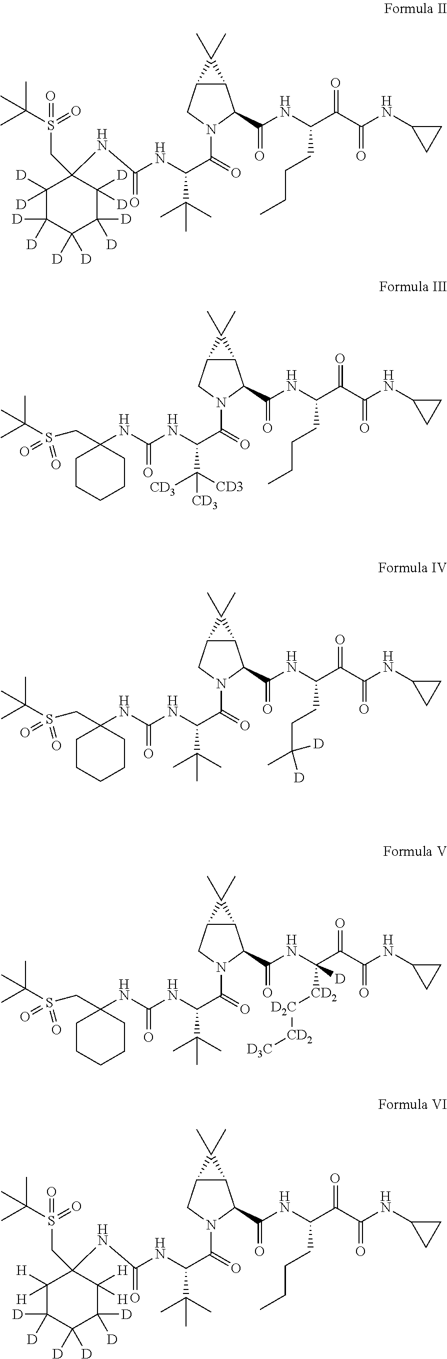

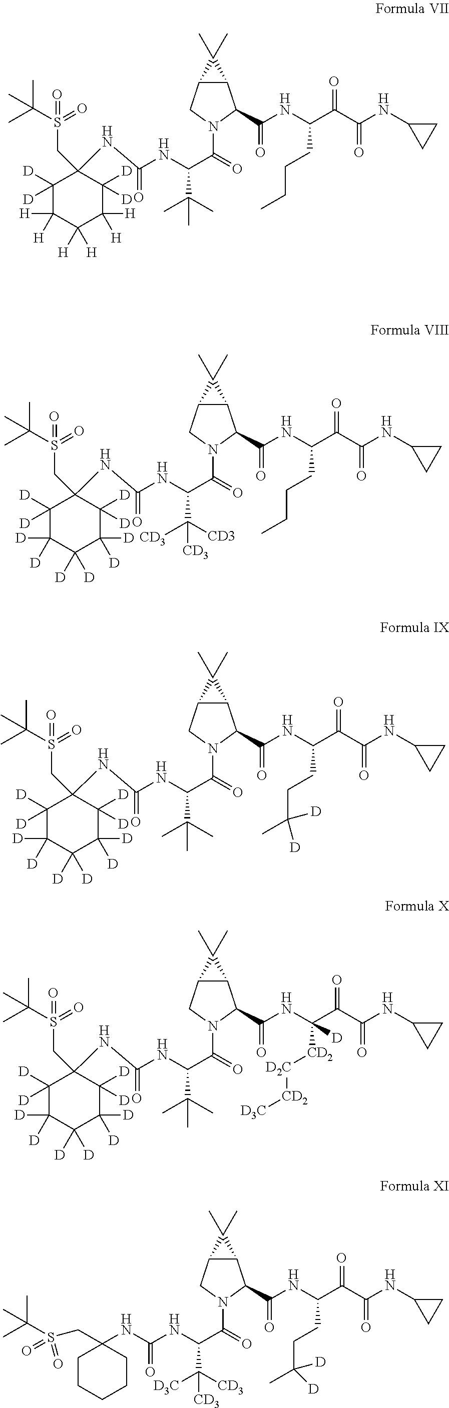

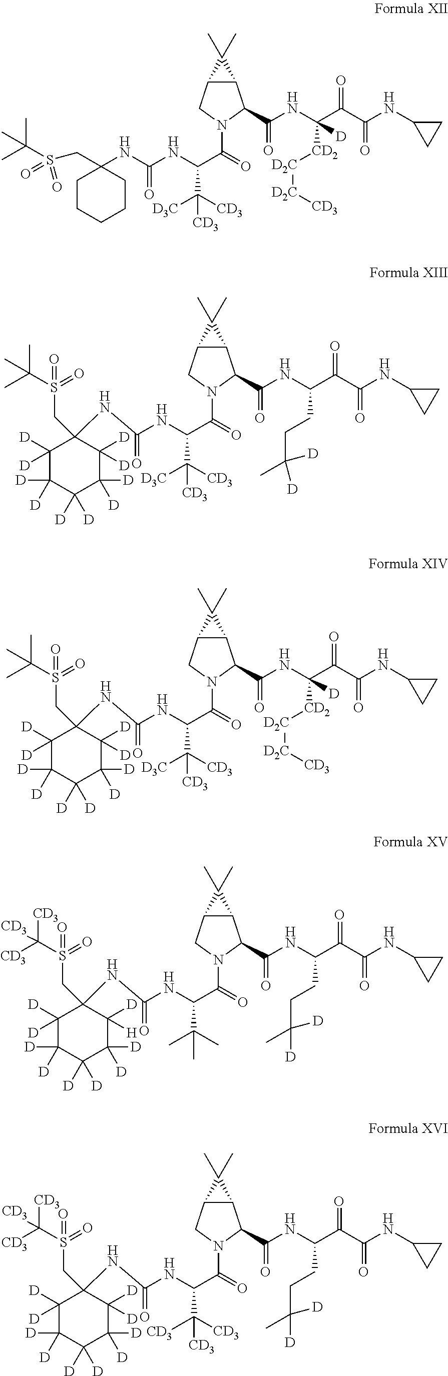

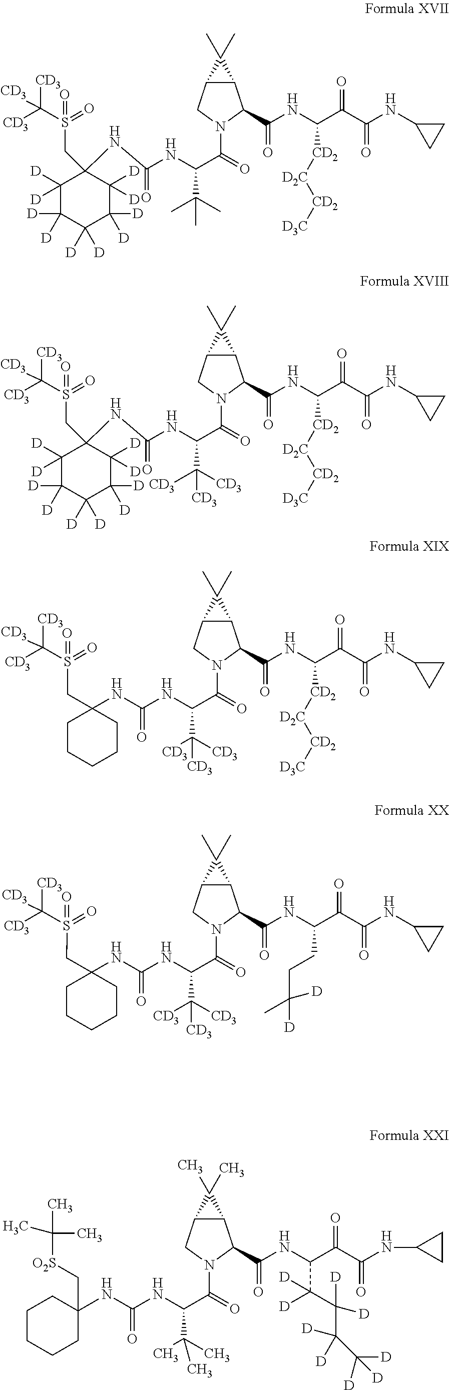

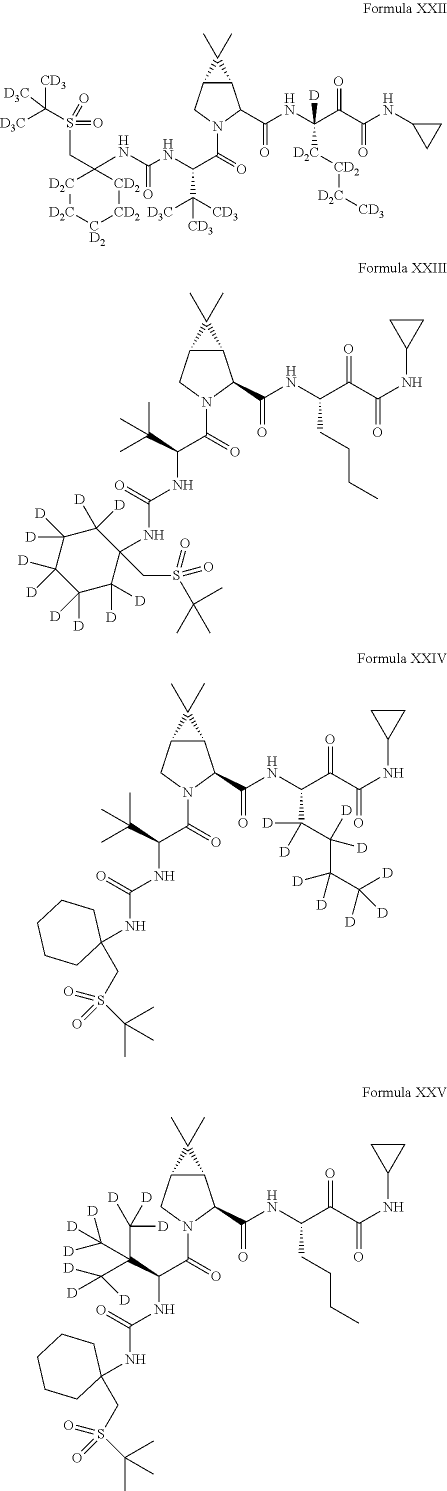

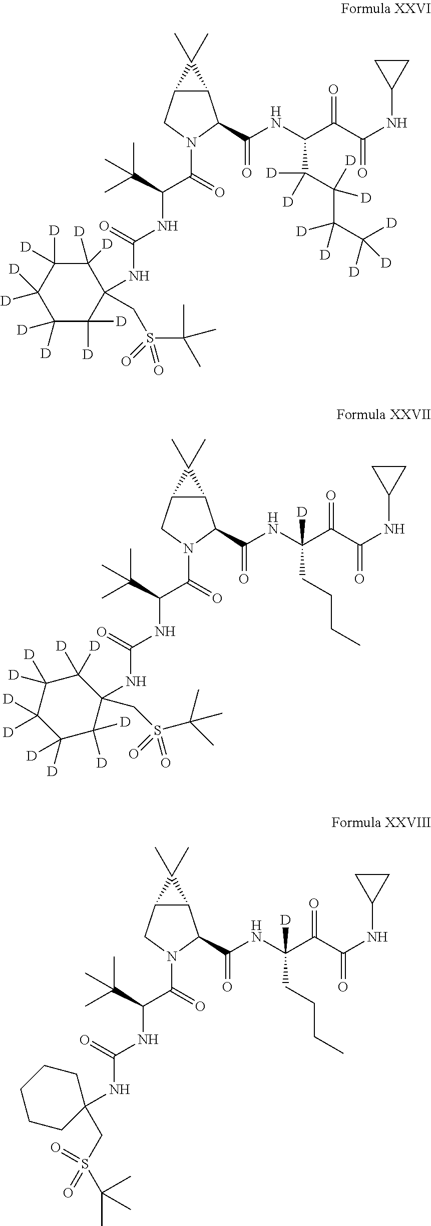

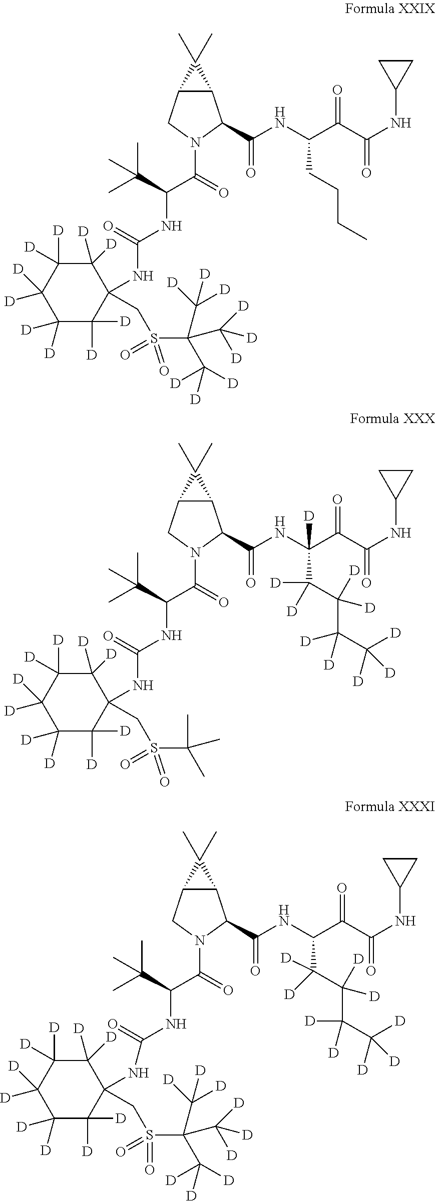

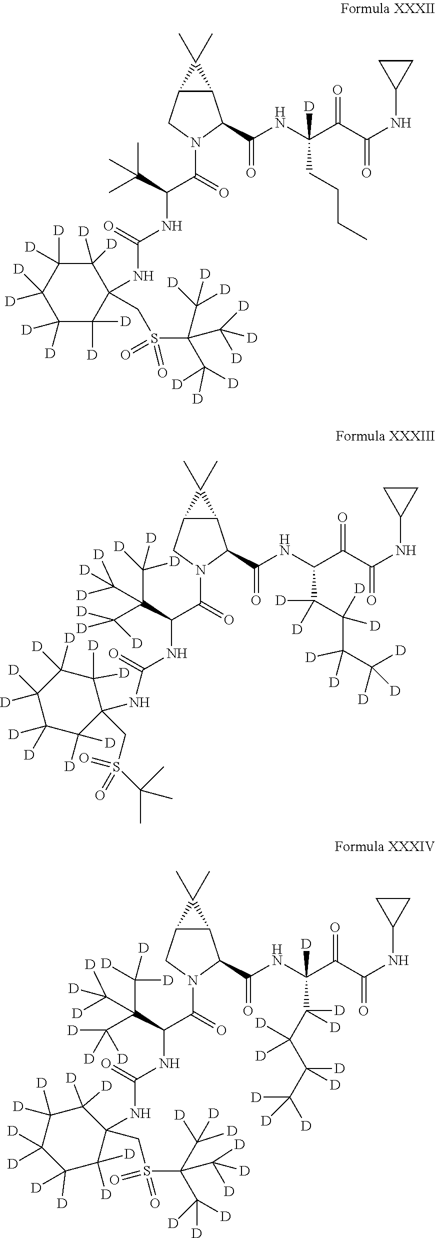

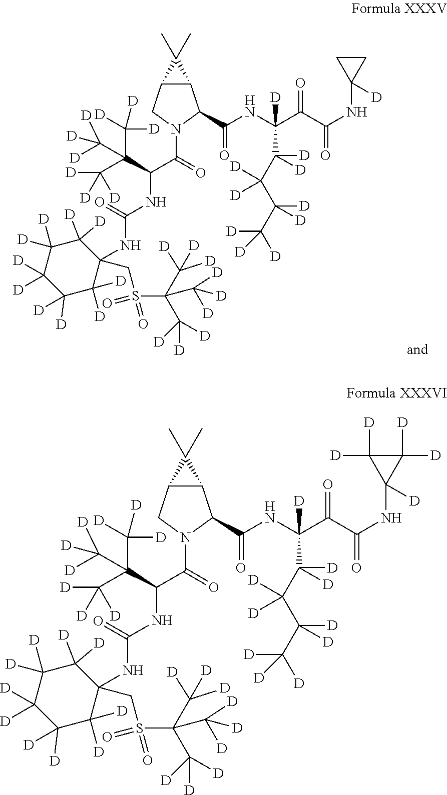

The term “Deuterated” in describing the compounds of this invention means that the deuterium-to-hydrogen ratio in the deuterated areas of the molecule substantially exceeds the naturally occurring deuterium-to-hydrogen ratio. Wikipedia (http://en.wikipedia.org/wiki/Deuterium) suggests that deuterium has a natural abundance in the oceans of Earth of approximately one atom in 6500 of hydrogen (˜154 PPM). Deuterium thus accounts for approximately 0.015% (on a weight basis, 0.030%) of all naturally occurring hydrogen in the oceans on Earth. However, other sources suggest a much higher abundance of e.g. 6·10⁻⁴ (6 atoms in 10,000 or 0.06% atom basis).

Deuteration of molecules and preparation of deuterated drugs are known. See, for example, M. Tanabe et al, “The Pharmacologic Effect of Deuterium Substitution on 5-n-Butyl-5-ethyl Barbituric Acid’, Life Sciences (1969) Vol. 8, part I, pp. 1123-1128; N. J. Haskins, “The Application of Stable Isotopes in Biomedical Research”, Biomedical Mass Spectrometry (1981), Vol. 9 (7), pp. 2690277; and the announcements from Concert Pharma (http://www.concertpharma.com/ConcertAnnouncesPreclinicalResultsiCAAC.htm) regarding preclinical results of their deuterated antibiotic, C-20081, and http://www.concertpharma.com/news/ConcertBeginsCTP347PhaseI.htm regarding Phase I clinical trials of their deuterium-containing serotonin modulator, CTP-347.

The compounds represented by Formulas II-XXXVI, by themselves or in combination with one or more other suitable agents disclosed herein, can be useful for treating diseases such as, for example, HCV, HIV, (AIDS, Acquired Immune Deficiency Syndrome), and related disorders, as well as for modulating the activity of hepatitis C virus (HCV) protease, preventing HCV, or ameliorating one or more symptoms of hepatitis C. Such modulation, treatment, prevention or amelioration can be done with the inventive compounds as well as with pharmaceutical compositions or formulations comprising such compounds. Without being limited to theory, it is believed that the HCV protease may be the NS3 or NS4a protease. The inventive compounds can inhibit such protease. They can also modulate the processing of hepatitis C virus (HCV) polypeptide.

This invention provides compounds of formulas II-XXXVI in pure, or isolated, or pure and isolated form.

This invention provides pharmaceutically acceptable salts of the compounds of formulas II-XXXVI.

This invention provides pharmaceutically acceptable esters of the compounds of formulas II-XXXVI.

This invention provides solvates of the compounds of formulas II-XXXVI.

This invention also provides a composition comprising an effective amount of at least one compound of formulas II-XXXVI.

This invention also provides a pharmaceutical composition comprising an effective amount of at least one compound of formulas II-XXXVI and a pharmaceutically acceptable carrier.

This invention also provides a pharmaceutical composition comprising an effective amount of at least one compound of formulas II-XXXVI and an effective amount of at least one other pharmaceutically active ingredient (such as, for example, a chemotherapeutic agent), and a pharmaceutically acceptable carrier.

As used above, and throughout this disclosure, the following terms, unless otherwise indicated, shall be understood to have the following meanings:

“Patient” includes both human and animals.

“Mammal” means humans and other mammalian animals.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

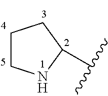

there is no —OH attached directly to carbons marked 2 and 5.

It should also be noted that tautomeric forms such as, for example, the moieties:

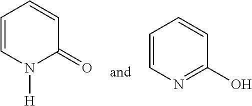

are considered equivalent in certain embodiments of this invention.

The term “one or more” or “at least one”, when indicating the number of substituents, compounds, combination agents and the like, refers to at least one, and up to the maximum number of chemically and physically permissible, substituents, compounds, combination agents and the like, that are present or added, depending on the context. Such techniques and knowledge are well known within the skills of the concerned artisan.

It should also be noted that any heteroatom with unsatisfied valences in the text, schemes, examples and Tables herein is assumed to have the hydrogen atom(s) to satisfy the valences.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of Formula I or a salt and/or solvate thereof. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

“Effective amount” or “therapeutically effective amount” is meant to describe an amount of compound or a composition of the present invention effective in inhibiting HCV and thus producing the desired therapeutic, ameliorative, inhibitory or preventative effect.

The compounds of Formulas II-XXXVI can form salts which are also within the scope of this invention. Reference to a compound of Formulas II-XXXVI herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formulas II-XXXVI contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formulas II-XXXVI may be formed, for example, by reacting a compound of Formulas II-XXXVI with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds of Formulas II-XXXVI, and salts, solvates, esters and prodrugs thereof, may exist in their tautomeric form (for example, as an amide or imino ether). All such tautomeric forms are contemplated herein as part of the present invention.

All stereoisomers (for example, geometric isomers, optical isomers and the like) of the present compounds (including those of the salts, solvates, esters and prodrugs of the compounds as well as the salts and solvates of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate” “prodrug” and the like, is intended to equally apply to the salt, solvate and prodrug of enantiomers, stereoisomers, rotamers, tautomers, positional isomers, racemates or prodrugs of the inventive compounds.

Polymorphic forms of the compounds of Formulas II-XXXVI, and of the salts, solvates, esters and prodrugs of the compounds of Formulas II-XXXVI, are intended to be included in the present invention.

It is to be understood that the utility of the compounds of Formulas II-XXXVI for the therapeutic applications discussed herein is applicable to each compound by itself or to the combination or combinations of one or more compounds of Formulas II-XXXVI as illustrated, for example, in the next immediate paragraph. The same understanding also applies to pharmaceutical composition(s) comprising such compound or compounds and method(s) of treatment involving such compound or compounds.

The compounds according to the invention can have pharmacological properties; in particular, the compounds of Formulas II-XXXVI can be inhibitors of HCV protease, each compound by itself or one or more compounds of Formulas II-XXXVI can be combined with one or more compounds selected from within Formulas II-XXXVI. The compound(s) can be useful for treating diseases such as, for example, HCV, HIV, (AIDS, Acquired Immune Deficiency Syndrome), and related disorders, as well as for modulating the activity of hepatitis C virus (HCV) protease, preventing HCV, or ameliorating one or more symptoms of hepatitis C.

The compounds of Formula I may be used for the manufacture of a medicament to treat disorders associated with the HCV protease, for example, the method comprising bringing into intimate contact a compound of Formula I a pharmaceutically acceptable carrier.

In another embodiment, this invention provides pharmaceutical compositions comprising the inventive compound or compounds as an active ingredient. The pharmaceutical compositions generally additionally comprise at least one pharmaceutically acceptable carrier diluent, excipient or carrier (collectively referred to herein as carrier materials). Because of their HCV inhibitory activity, such pharmaceutical compositions possess utility in treating hepatitis C and related disorders.

In yet another embodiment, the present invention discloses methods for preparing pharmaceutical compositions comprising the inventive compounds as an active ingredient. In the pharmaceutical compositions and methods of the present invention, the active ingredients will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition.

Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum and the like.

Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. HCV inhibitory activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injections or addition of sweeteners and pacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool and thereby solidify.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions may take the form of creams, lotions, aerosols and/or emulsions and can be included in a transdermal patch of the matrix or reservoir type as are conventional in the art for this purpose.

The compounds of the invention may also be administered orally, intravenously, intranasally or subcutaneously.

The compounds of the invention may also comprise preparations which are in a unit dosage form. In such form, the preparation is subdivided into suitably sized unit doses containing appropriate quantities of the active components, e.g., an effective amount to achieve the desired purpose.

The quantity of the inventive active composition in a unit dose of preparation may be generally varied or adjusted from about 1.0 milligram to about 1,000 milligrams, preferably from about 1.0 to about 950 milligrams, more preferably from about 1.0 to about 500 milligrams, and typically from about 1 to about 250 milligrams, according to the particular application. The actual dosage employed may be varied depending upon the patient's age, sex, weight and severity of the condition being treated. Such techniques are well known to those skilled in the art.

Generally, the human oral dosage form containing the active ingredients can be administered 1 or 2 times per day. The amount and frequency of the administration will be regulated according to the judgment of the attending clinician. A generally recommended daily dosage regimen for oral administration may range from about 1.0 milligram to about 1,000 milligrams per day, in single or divided doses.

Some useful terms are described below:

Capsule—refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet—refers to a compressed or molded solid dosage form containing the active ingredients with suitable diluents. The tablet can be prepared by compression of mixtures or granulations obtained by wet granulation, dry granulation or by compaction.

Oral gel—refers to the active ingredients dispersed or solubilized in a hydrophillic semi-solid matrix.

Powder for constitution refers to powder blends containing the active ingredients and suitable diluents which can be suspended in water or juices.

Diluent—refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

Disintegrant—refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

Binder—refers to substances that bind or “glue” powders together and make them cohesive by forming granules, thus serving as the “adhesive” in the formulation. Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant—refers to a substance added to the dosage form to enable the tablet, granules, etc. after it has been compressed, to release from the mold or die by reducing friction or wear. Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d′l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glident—material that prevents caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents—excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

Bioavailability—refers to the rate and extent to which the active drug ingredient or therapeutic moiety is absorbed into the systemic circulation from an administered dosage form as compared to a standard or control.

Conventional methods for preparing tablets are known. Such methods include dry methods such as direct compression and compression of granulation produced by compaction, or wet methods or other special procedures. Conventional methods for making other forms for administration such as, for example, capsules, suppositories and the like are also well known.

Another embodiment of the invention discloses the use of the inventive compounds or pharmaceutical compositions disclosed above for treatment of diseases such as, for example, hepatitis C and the like. The method comprises administering a therapeutically effective amount of the inventive compound or pharmaceutical composition to a patient having such a disease or diseases and in need of such a treatment.

In yet another embodiment, the compounds of the invention may be used for the treatment of HCV in humans in monotherapy mode or in a combination therapy (e.g., dual combination, triple combination etc.) mode such as, for example, in combination with antiviral and/or immunomodulatory agents. Examples of such antiviral and/or immunomodulatory agents include Ribavirin (from Schering-Plough Corporation, Madison, N.J. ) and Levovirin™ (from ICN Pharmaceuticals, Costa Mesa, Calif.), VP 50406™ (from Viropharma, Incorporated, Exton, Pa.), ISIS14803™ (from ISIS Pharmaceuticals, Carlsbad, Calif.), Heptazyme™ (from Ribozyme Pharmaceuticals, Boulder, Colo.), VX 497™ (from Vertex Pharmaceuticals, Cambridge, Mass.), Thymosin™ (from SciClone Pharmaceuticals, Sari Mateo, Calif.), Maxamine™ (Maxim Pharmaceuticals, San Diego, Calif.), mycophenolate mofetil (from Hoffman-LaRoche, Nutley, N.J.), interferon (such as, for example, interferon-alpha, PEG-interferon alpha conjugates) and the like. “PEG-interferon alpha conjugates” are interferon alpha molecules covalently attached to a PEG molecule. Illustrative PEG-interferon alpha conjugates include interferon alpha-2a (Roferon™, from Hoffman La-Roche, Nutley, N.J.) in the form of pegylated interferon alpha-2a (e.g., as sold under the trade name Pegasys™), interferon alpha-2b (Intron™, from Schering-Plough Corporation) in the form of pegylated interferon alpha-2b (e.g., as sold under the trade name PEG-Intron™), interferon alpha-2c (Berofor Alpha™, from Boehringer Ingelheim, Ingelheim, Germany) or consensus interferon as defined by determination of a consensus sequence of naturally occurring interferon alphas (Infergen™, from Amgen, Thousand Oaks, Calif.).

As stated earlier, the invention includes tautomers, rotamers, enantiomers and other stereoisomers of the inventive compounds also. Thus, as one skilled in the art appreciates, some of the inventive compounds may exist in suitable isomeric forms. Such variations are contemplated to be within the scope of the invention.

Another embodiment of the invention discloses a method of making the compounds disclosed herein. The compounds may be prepared by several techniques known in the art. Illustrative procedures are outlined in the following reaction schemes. The illustrations should not be construed to limit the scope of the invention which is defined in the appended claims. Alternative mechanistic pathways and analogous structures will be apparent to those skilled in the art.

It is to be understood that while the following illustrative schemes describe the preparation of a few representative inventive compounds, suitable substitution of any of both the natural and unnatural amino acids will result in the formation of the desired compounds based on such substitution. Such variations are contemplated to be within the scope of the invention.

For the procedures described below, the following abbreviations are used:

### THF: Tetrahydrofuran

### DMF: N,N-Dimethylformamide

EtOAc: Ethyl acetate
AcOH: Acetic acid
HOOBt: 3-Hydroxy-1,2,3-benzotriazin-4(3H)-one
EDCI: 1-(3-dimethylaminopropyl)-3-ethylcarbodiimide hydrochloride

### NMM: N-Methylmorpholine

### ADDP: 1,1′-(Azodicarbobyl)dipiperidine

### DEAD: Diethylazodicarboxylate

### MeOH: Methanol

### EtOH: Ethanol

Et2O: Diethyl ether

### DMSO: Dimethylsulfoxide

### HOBt: N-Hydroxybenzotriazole

PyBrOP: Bromo-tris-pyrrolidinophosphonium hexafluorophosphate

### DCM: Dichloromethane

### DCC: 1,3-Dicyclohexylcarbodiimide

TEMPO: 2,2,6,6-Tetramethyl-1-piperidinyloxy

### Phg: Phenylglycine

### Chg: Cyclohexylglycine

### Bn: Benzyl

### Bzl: Benzyl

### Et: Ethyl

### Ph: Phenyl

DMF-DMA: N,N-Dimethylformamide-dimethylacetal iBoc: isobutoxycarbonyl iPr: isopropyl
^{t}Bu or Bu^{t}: tert-Butyl
Boc: tert-Butyloxycarbonyl

### Cbz: Benzyloxycarbonyl

### Cp: Cylcopentyldienyl

Ts: p-toluenesulfonyl

### Me: Methyl

HATU: O-(7-azabenzotriazol-1-yl)-1,1,3,3-tetramethyluronium hexafluorophosphate

### DMAP: 4-N,N-Dimethylaminopyridine

BOP: Benzotriazol-1-yl-oxy-tris(dimethylamino)hexafluorophosphate

### PCC: Pyridiniumchlorochromate

KHMDS: Potassium Hexamethyldisilazide or Potassium bis(trimethylsilylamide)
NaHMDS: Sodium Hexamethyldisilazide or Sodium bis(trimethylsilylamide)
LiHMDS: Lithium Hexamethyldisilazide or Lithium bis(trimethylsilylamide) 10% Pd/C: 10% Palladium on carbon (by weight).

### TG: Thioglycerol

### General Schemes for Preparation of Target Compounds

The synthesis of the compounds of the present invention is exemplified by the synthesis of the compound of Formula II described below:

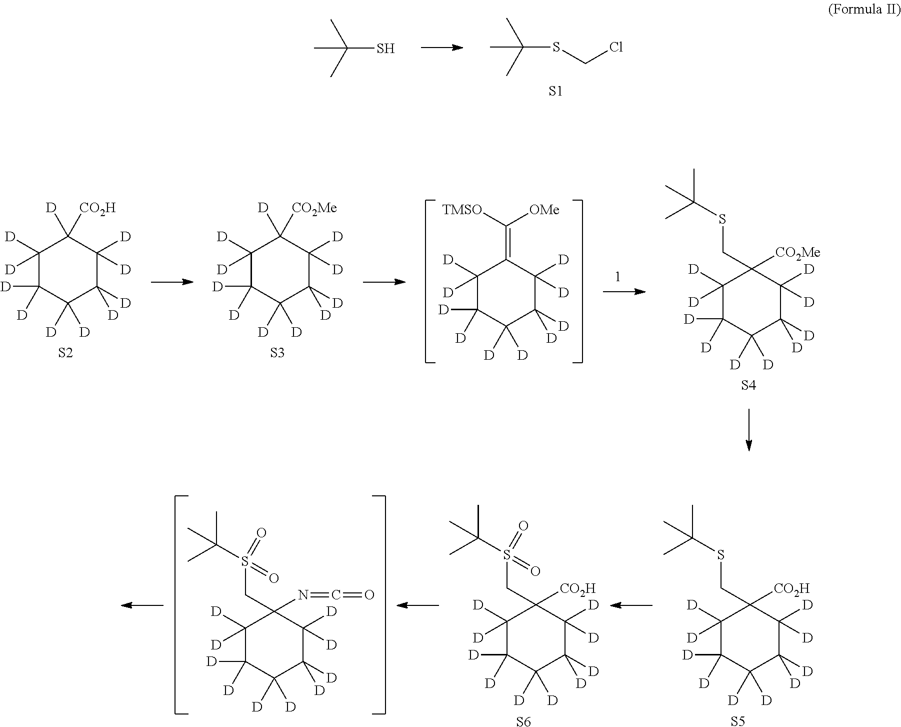

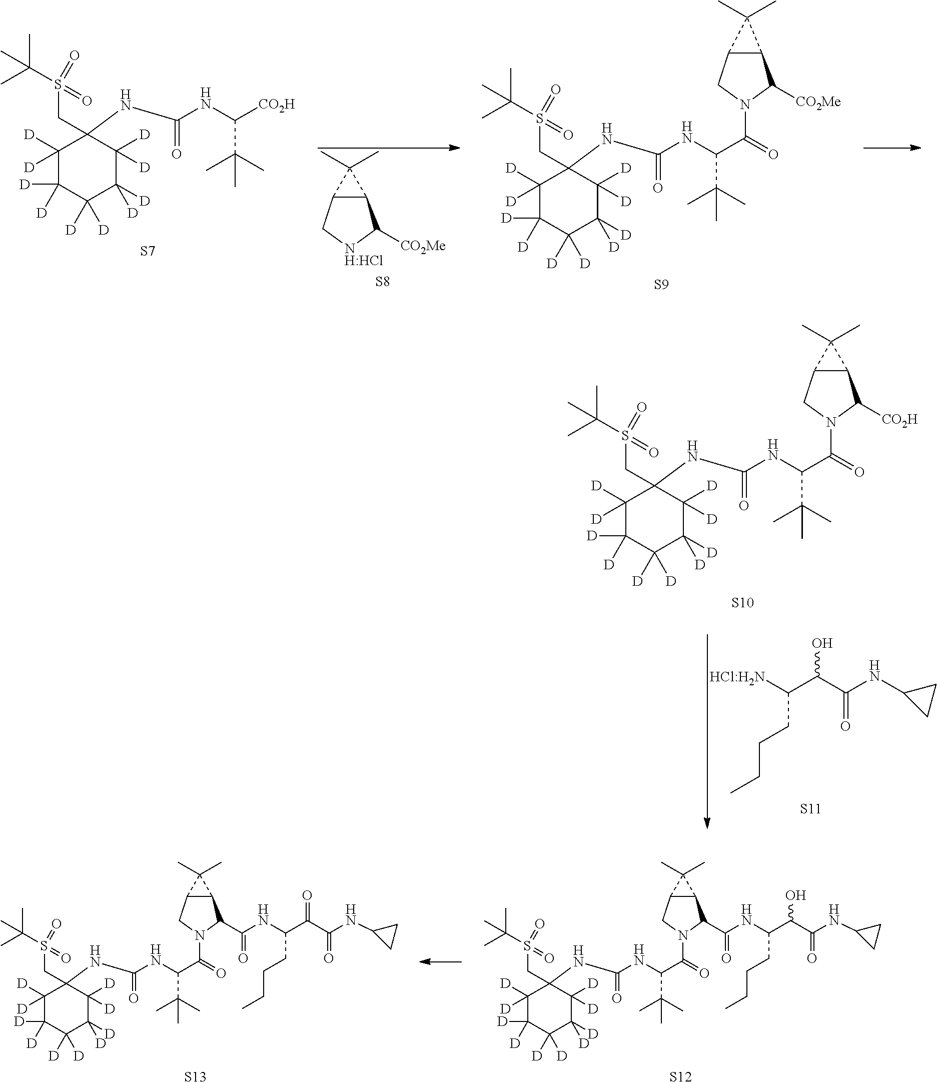

### Step 1:

A mixture of paraformaldehyde (6.00 g) and trimethylsilyichloride (32 ml) was cooled to 0 C and 2-methyl-2-propane thiol (22.5 ml) was added over a period of 3 h., via a syringe pump, under an atmosphere of nitrogen. The resulting mixture was stirred at 0° C. for 15 min. and then room temperature for 1.5 h. Magnesium sulfate (3.30 g) was added and the suspension was stirred for a further 15 min. and the filtered. The filtrate was distilled under reduced pressure to yield 1, which was kept at −20 C before use.

### Step 2:

The cyclohexane carboxylic acid (2 from DCN isotope lot # 03958 MH; 4.90 g) was dissolved in anhydrous dichloromethane (11.2 ml) and anhydrous methanol (4.65 ml) followed by conc. sulfuric acid (0.15 ml) were added and the resulting mixture was heated to 5° C., overnight. After cooling, the reaction was washed with water, sat. aqueous sodium bicarbonate, dried (sodium sulfate). The volatiles were removed under reduced pressure to yield a residue which was purified by distillation to give the methyl ester (3; 4.60 g).

### Step 3:

n-Butyllithium (8.5 ml of a 2.1M solution in hexanes) was added, over a period of 30 min., to a solution of diisopropylamine (2.50 ml) in anhydrous THF (5.4 ml) at −30 C, under an atmosphere of argon. When the addition was complete, the mixture was stirred at −10 C for 1 h and the cooled to −78 C. The methyl ester (3; 2.74 g) in anhydrous THF (20 ml) was added over a period of 108 min. The mixture was allowed to warm to −50 C over a period of 1 h. and then cooled again to −78 C before the addition of trimethylsilylchloride (2.3 ml) over a period of 30 min. The reaction was stirred at −78° C. for 30 min., -10° C. for 1 h and room temperature for 30 min. The volatiles were removed under reduced pressure to yield a solid. Anhydrous dichloromethane (17.6 ml) was added to form a white suspension. A solution of 3 (2.48 g) in anhydrous dichloromethane (4.4 ml) was added (over 20 min) followed by zinc bromide (0.403 g) and the resulting reaction mixture was stirred at room temperature, overnight. The reaction was transferred to a separating funnel and washed with sat. aqueous sodium bicarbonate, water, dried (sodium sulfate). The volatiles were removed under reduced pressure to give the desired sulfide (4; 4.38 g), as a yellow oil.

### Step 4:

Lithium hydroxide (15 g) was added to a solution of the methyl ester (4; 6.91 g) in a mixture of methanol (20.5 ml), THF (34 ml) and water (20.5 ml) and the resulting mixture was heated to reflux for 2 days. After cooling, the volatiles were removed under reduced pressure. Water (5 ml) was added to the residue and acidified to pH=2 with 3N HCl. The organics were extracted into EtOAc (X3). The combined organic phases were washed with water, brine, dried (sodium sulfate). The volatiles were removed under reduced pressure to give the carboxylic acid (5; 6.00 g).

### Step 5:

A suspension of oxone (36.1 g) in water (51.6 ml) was added to a methanol (72 ml) solution of the sulfide (5; 6.00 g) and the resulting mixture was stirred at room temperature overnight. The reaction was filtered and the solid was washed thoroughly with methanol. the combined filtrate was concentrated to dryness under reduced pressure. The solid was dissolved in EtOAc and washed with water, dried (sodium sulfate) and concentrated to give the sulfone (6; 5.80 g).

### Step 6:

Diphenylphosphorylazide (1.6 ml; 1 eq) was added to a solution of triethylamine (3.1 ml; 3 eq.) and the carboxylic acid (6; 2.0 g; 1 eq.) in anhydrous toluene (20 ml) and the mixture was heated to 8° C. for 4 h. After cooling to room temperature, sat. aqueous sodium bicarbonate was added. The organic phase was separated, washed with a further portion of saturated aqueous sodium bicarbonate and the toluene solution set aside.

L-tert-Leucine (0.963 g), water (8.0 ml) and triethylamine (3.3 ml) were mixed, cooled to 0 C, and the aforementioned toluene solution was added in one portion. The mixture was stirred at 0 C for 5 min and then room temperature overnight. Aqueous sodium carbonate (10%; 14 ml) was added and the mixture was stirred for 10 min. The aqueous phase was separated, acidified to pH=1.5 with 3N HCl. The precipitate was collected, washed with water and dried overnight. Gave the desired urea (7; 1.45 g). A further portion of urea (0.288 g) was obtained from the resulting filtrate, on standing.

### Step 7:

EDCI (3.22 g) was added to a mixture of the carboxylic acid (7; 5.38 g), the hydrochloride salt (8; 3.12 g), N-methylmorpholine (2.20 ml) and HOBT (0.363 g) in anhydrous acetonitrile (51.4 ml) at 0 C. The resulting reaction mixture was allowed to warm to room temperature, overnight. The reaction was partitioned between EtOAc and aqueous 1N HCl. The organic phase was separated, washed with saturated aqueous sodium bicarbonate, 10% aqueous potassium carbonate solution, brine and then concentrated to dryness to provide the desired amide (9; 7.30 g), as a clear foam.

### Step 8:

An aqueous solution of lithium hydroxide (1 M; 39.8 ml) was added to a solution of the methyl ester (9; 7.30 g) in a mixture of THF (37.8 ml) and methanol (37.8 ml) at 0 C. The resulting mixture was stirred at 0 C for 2 h., then at room temperature for 3 h. The volatiles were removed under reduced pressure. Water (30 ml) and EtOAc (60 ml) were added to the residue and the mixture was stirred for 10 min. The aqueous phase was separated and acidified to pH=1 with 3N HCl and the organics were extracted into EtOAc. The combined EtOAc phases were washed with brine, dried (sodium sulfate) and the volatiles were removed under reduced pressure to give the desired carboxylic acid (10, 6.74 g).

### Step 9:

EDCI (2.65 g) was added to a mixture of the carboxylic acid (10; 6.20 g), hydrochloride salt (11; 3.00 g), HOBT (0.312 g) and diisopropylethylamine (2.88 ml) in anhydrous acetonitrile 115 ml) at 0 C. The resulting mixture was stirred at 0 C for 5 min. and at room temperature overnight. The volume of the reaction was reduced to @50 ml under reduced pressure and EtOAc (100 ml) was added. The mixture was washed with 1N HCl (aqueous, sat aqueous sodium bicarbonate, 10% aqueous potassium carbonate, brine, dried (sodium sulfate) and the volatiles were removed under reduced pressure. The residue was purified using an ISCO column with dichloromethane; methanol (95;5) as eluent to give the desired amide (12; 6.80 g).

### Step 10:

The Dess-Martin periodinane (7.90 g) was added to a solution of the alcohol (12; 6.80 g) in anhydrous dichloromethane (35 ml) and the resulting mixture was stirred at room temperature for 2 h. Diethyl ether (200 ml) followed by sat. aqueous sodium bicarbonate containing sodium thiosulfate were added and stirring was continued for a further 5 min. Water (100 ml) was added and the organic phase was separated and washed with sat. aqueous sodium bicarbonate, water, dried (sodium sulfate) and the volatiles were removed under reduced pressure. The residue was purified using an ISCO column using hexanes to EtOAc as eluent to give the ketoamide (13; 5.76 g). The product was further purified by crystallization (acetone/water) to give the ketoamide (13; 3.80 g), as a white powder.

Syntheses of some other compounds of the invention are exemplified below.

### Synthesis of [D₉]-(1R,5S)-methyl 3-((S)-(tert-butoxycarbonylamino -3,3-dimethylbutanoyl)-6,6-dimethyl-3-azabicyclo[3.1.0]hexane-2-carboxylate [1]

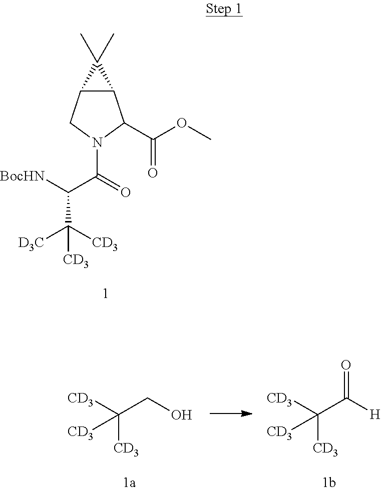

A solution of [1a] [D₉]-2,2-dimethylpropan-1-ol (1.00 g, 8.83 mmol) in ether (20 mL) and CHCl₃ (20 mL) was treated with pyridinium chlorochromate (2.00 g, 9.27 mmol) and stirred at rt. for 12 h. The reaction mixture was filtered through a plug of celite and washed with small amount of CHCl₃, and the filtrate was directly used in next reaction.

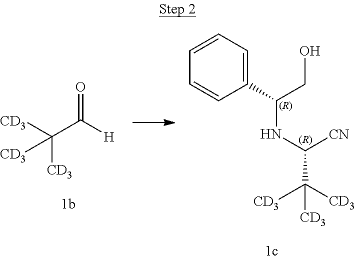

[D₉]-Trimethylacetaldehyde (1b, 1.00 g, 8.99 mmol), (R)-(−)-2-phenylglycinol (1.23 g, 8.99 mmol) and 4A molecular sieves (2.5 g) was treated with trimethylsilyl cyanide (2.40 mL, 18.0 mmol) and stirred overnight. The reaction was then treated with tetra-n-butylammonium fluoride (4.70 g, 18.0 mmol) and stirred for 0.5 h. The reaction mixture was concentrated in vacuo, diluted with EtOAc (200 mL) and washed extensively with water. The combined organic layer was dried (MgSO₄), filtered, concentrated in vacuo and purified by chromatography to yield [D₉]-(S)-2-((R)-2-hydroxy-1-phenylethylamino)-3,3-dimethylbutanenitrile 1c (460 mg; Yield=20%).

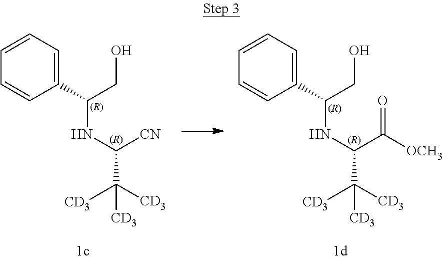

A solution of [1c] [D₉]-(S)-2-((R)-2-hydroxy-1-phenylethylamino)-3,3-dimethylbutanenitrile (460 mg, 1.8 mmol) in 6 M of hydrogen chloride in methanol (10.00 mL) was heated at reflux for 12 h and concentrated in vacuo. After concentrating, the reaction was repeated once again and the crude product was purified by chromatography SiO₂ (EtOAc/Hexanes) to yield [1d] [D₉]-(S)-methyl 2-((R)-2-hydroxy-1-phenylethylamino)-3,3-dimethylbutanoate (300 mg; Yield=60%) as an inseparable mixture of diastereomers.

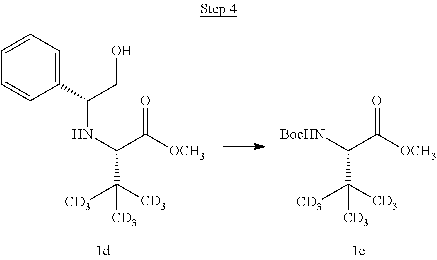

A solution of [1d] [D₉]-(S)-methyl 2-((R)-2-hydroxy-1-phenylethylamino)-3,3-dimethylbutanoate (150 mg, 0.55 mmol) in methanol (10.00 mL, 246.9 mmol) was treated with 20% palladium hydroxide on carbon (1:4, palladium hydroxide:carbon black, 150 mg) and hydrogenated at 50 psi. The reaction mixture was filtered through a plug of celite. The filtrate was concentrated in vacuo and stirred with Boc₂O in CH₂Cl₂ the residue was purified by chromatography to yield [1e] [D]₉—(S)-methyl 2-(tert-butoxycarbonylamino)-3,3-dimethylbutanoate (125 mg; Yield=90%) as a colorless oil.

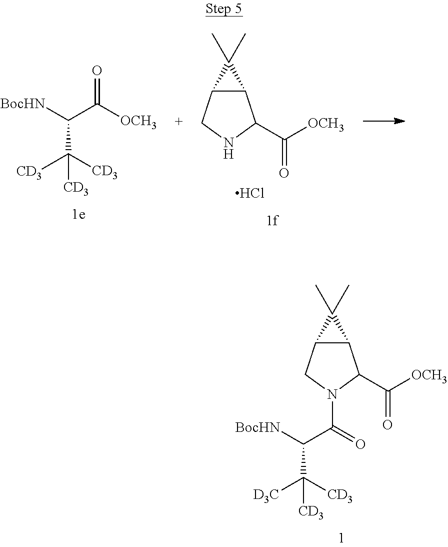

Ester 1e (125 mg, 0.50 mmol) in methanol, THF and water (3 mL each) was treated with LiOH.H₂O (100 mg) and stirred at rt for 4 h. The disappearance of starting material was followed by TLC. On completion of the reaction the reaction mixture was acidified with aq HCl (1 M) and extracted into EtOAc (3×30 mL). The combined organic layer was dried (MgSO₄) filtered and concentrated in vacuo to yield [D]₉—(R)-2-(tert-butoxycarbonylamino)-3,3-dimethylbutanoic acid that was used in the next reaction without purification.

A solution of the acid [D]₉—(R)-2-(tert-butoxycarbonylamino)-3,3-dimethylbutanoic acid (120.0 mg, 0.499 mmol;) in N,N-Dimethylformamide (3.00 mL, 38.7 mmol) and methylene chloride (3.00 mL, 46.8 mmol) at 0° C. was treated with 4-methylmorpholine (137 μL, 1.24 mmol), N,N,N′,N′-tetramethyl-O-(7-azabenzotriazol-1-yl)uronium hexafluorophosphate (0.284 g, 0.74892 mmol; Supplier=Aldrich) and [3f] (1R,2S,5S)-methyl-6,6-dimethyl-3-azabicyclo[3.1.0]hexane-2-carboxylate hydrochloride (0.154 g, 0.74 mmol) and stirred at freezer overnight. The reaction mixture was diluted with EtOAc (100.00 mL) and washed with aq. sodium bicarbonate, aq HCl and brine. The combined organic layers were dried (MgSO₄), filtered concentrated in vacuo and purified by chromatography (SiO₂, EtOAc/Hexanes) to yield [1] [D₉]-(1R,2S,5S)-methyl 3-((R)-2-(tert-butoxycarbonylamino)-3,3-dimethylbutanoyl)-6,6-dimethyl-3-azabicyclo[3.1.0]hexane-2-carboxylate (145 mg; Yield=74.2%).

### Synthesis of deuterated-1-(tert-butylsulfonylmethyl)-1-isocyanatocyclohexane-(D10) (2)

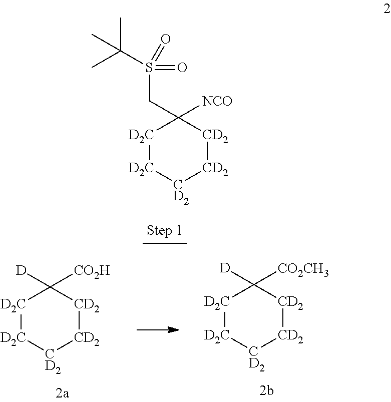

[D₁₁]-cyclohexanecarboxylic acid (2a, 5.0 g, 36 mmol) was dissolved in anhydrous MeOH (5 mL) and anhydrous CH₂Cl₂ (12 mL). H₂SO₄ (0.2 mL) was added and the solution was stirred at 50° C. for 24 h under N₂. After cooling down, water (20 mL) and CH₂Cl₂ (50 ml) were added. The aqueous layer was separated, extracted with CH₂Cl₂ (20 ml) once. The CH₂Cl₂ layers were combined, washed with saturated NaHCO₃ (40 ml), brine (40 ml), dried over anhydrous Na₂SO₄, filtered, and concentrated. The product was dried under vacuum for 20 minutes to give 2b (5.12 g, 93%).

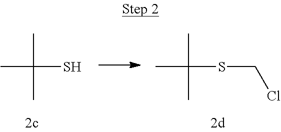

To a flame-dried 250 mL round bottom flask was added chlorotrimethylsilane (32.0 mL, 250 mmol) and paraformaldehyde (6.0 g, 200 mmol). The flask was cooled to 0° C. 2-Methyl-2-propanethiol (22.5 mL, 200 mmol) was added dropwise via an additional funnel. The solution was stirred at 0° C. for 10 min, then at room temperature for 1 h. The solid was removed by filtration. The product was purified by distillation to give 2d (18.5 g, 53%).

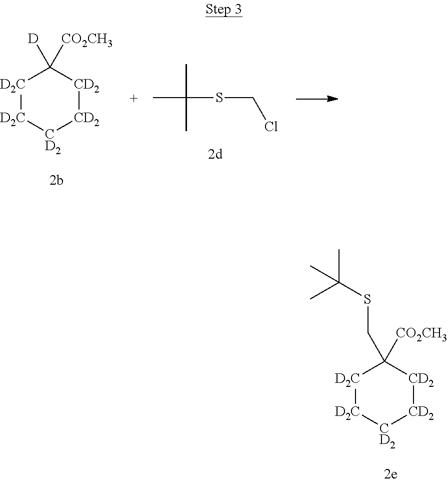

A solution of 2b (2.48 g, 16.2 mmol) in dry THF (60 mL) in a 250 mL round bottom flask was cooled to −78° C. LDA (2M in hexane, 9.7 mL, 19.4 mmol) was added dropwise. The solution was stirred at −50° C. for 1 h, and then chlorotrimethylsilane (2.48 mL, 19.4 mmol) was added dropwise at −78° C. After the solution was stirred at −78° C. for 20 min, it was allowed to warm to 0° C. in 30 min and then stirred at room temperature for 20 min. The solvent was removed under vacuum at 40° C. and dried under vacuum for 20 min. The residue was dissolved in anhydrous CH₂Cl₂ (60 mL) and 1d (2.69 g, 19.4 mmol, in 15 mL anhydrous CH₂Cl₂) was added dropwise, followed by ZnBr₂ (364 mg, 1.62 mmol) in one portion. The solution was stirred at room temperature for overnight. Water (50 mL) and CH₂Cl₂ (100 mL) were added. The aqueous layer was separated and extracted with CH₂Cl₂ (50 mL) once. The CH₂Cl₂ layers were combined, washed with saturated NaHCO₃ (80 ml), brine (80 ml), dried over anhydrous Na₂SO₄, filtered, and concentrated. The product was dried under vacuum for 3 h to give 2e (4.14 g) which was used without further purification.

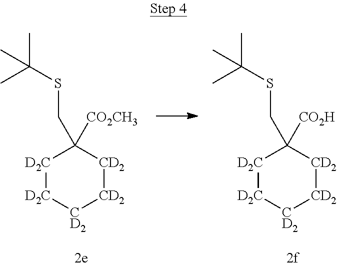

To a 350 pressure bottle was added 2e (4.00 g, 15/mmol), MeOH (34 mL), THF (20 mL), water (20 ml), and LiOH monohydrate (1.98 g, 47.2 mmol). The bottle was sealed and stirred at 75° C. for 3 days. After cooling down, the solution was transferred into a 500 mL round bottom flask and the solvent was removed under vacuum. The residue was dissolved in HCl (2N, 100 mL) and EtOAc (150 ml). The aqueous layer was separated and extracted with EtOAc (50 mL) once. The EtOAc layers were combined, washed with brine (100 ml), dried over anhydrous Na₂SO₄, filtered, and concentrated. The product was dried under vacuum for overnight to give 2f (3.70 g, 98%).

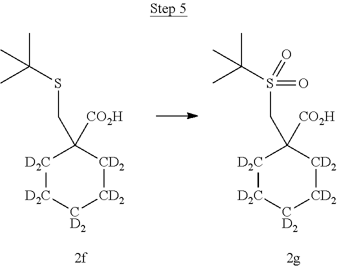

Oxone (23.7 g, 38.5 mmol) was suspended in water (35 mL) and was added into a solution of 2f (3.70 g, 15.4 mmol) in MeOH (50 mL). The solution was stirred at room temperature for overnight. The solid was removed by filtration and washed with MeOH (20 mL×3). The filtrate was concentrated. The residue was dissolved in EtOAc (250 mL) and washed with water (100 mL×2) and brine (100 mL). It was then dried over anhydrous Na₂SO₄, filtered, concentrated and dried under vacuum to give 2 g (2.95 g, 70.3%).

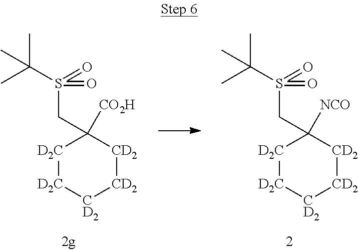

To a flamed-dried flask was added 2 g (200 mg, 0.734 mmol) and anhydrous toluene (2 mL). The solution was heated to 80° C. Diphenylphosphoryl azide (0.158 mL, 0.734 mmol) and triethylamine (0.102 mL, 0.734 mmol) were added at 80° C. The solution was stirred at 110° C. for 4 h. After cooling down, saturated NaHCO₃ (2 mL) was added. The aqueous layer was separated and extracted with toluene (1 ml). The toluene solution was combined and used without further purification.

### Synthesis of deuterated-1-(tert-butylsulfonylmethyl-(D19))-1-isocyanatocyclohexane-(D10) (structure 3)

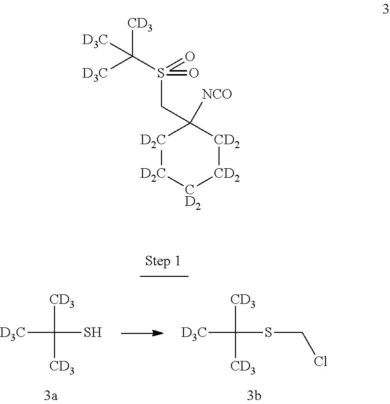

To a flame-dried 100 mL round bottom was added chlorotrimethylsilane (8.0 mL, 63.0 mmol) and paraformaldehyde (1.52 g, 50.4 mmol). The flask was cooled to 0° C. 2-methyl-2-propane-D₉-thiol (5.0 g, 50.4 mmol) was added dropwise via an additional funnel. The solution was stirred at 0° C. for 10 min, then at room temperature for 2 h. The product was purified by distillation to give 3b (1.9 g, 25%).

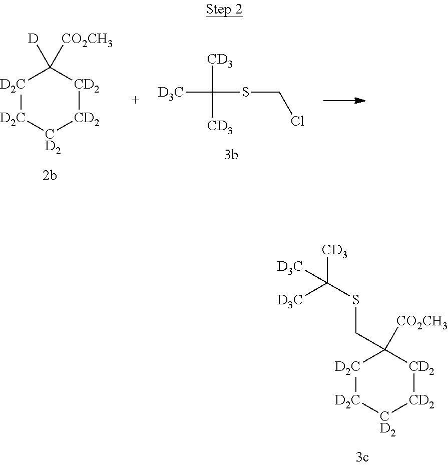

A solution of 2b (2.74 g, 17.9 mmol) in dry THF (60 mL) in a 250 mL round bottom flask was cooled to −78° C. LDA (2M in hexane, 10.7 mL, 21.4 mmol) was added dropwise. The solution was stirred between −78° C. and -50° C. for 1 h, and then chlorotrimethylsilane (2.74 mL, 21.4 mmol) was added dropwise at −78° C. After the solution was stirred at −78° C. for 20 min, it was allowed to warm to 0° C. in 30 min and then stirred at room temperature for 20 min. The solvent was removed under vacuum at 40° C. water bath and dried under vacuum for 20 min. The residue was dissolved in anhydrous CH₂Cl₂ (60 mL) and 3b (2.4 g, 16.3 mmol, in 15 mL anhydrous CH₂Cl₂) was added dropwise, followed by ZnBr₂ (405 mg, 1.8 mmol) in one portion. The solution was stirred at room temperature for overnight. Water (50 mL) and CH₂Cl₂ (50 mL) were added. The aqueous layer was separated and extracted with CH₂Cl₂ (20 mL) once. The CH₂Cl₂ layers were combined, washed with saturated NaHCO₃ (30 ml), brine (30 ml), dried over anhydrous Na₂SO₄, filtered, and concentrated. The product was dried under vacuum for 3 h to give 3c (4.61 g, 98%) which was used without further purification.

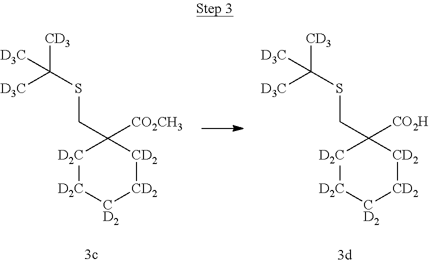

To a 350 pressure bottle was added 3c (4.28 g, 16.3 mmol), MeOH (35 mL), THF (20 mL), water (20 ml), and LiOH monohydrate (2.00 g, 48.7 mmol). The bottle was sealed and stirred at 75° C. for 3 days. After cooling down, the solution was transferred into a 500 mL round bottom flask and the solvent was removed under vacuum. The residue was dissolved in HCl (2N, 100 mL) and EtOAc (150 ml). The aqueous layer was separated and extracted with EtOAc (50 mL) once. The EtOAc layers were combined, washed with brine (100 ml), dried over anhydrous Na₂SO₄, filtered, and concentrated. The product was dried under vacuum for overnight to give 3d (3.84 g, 94%).

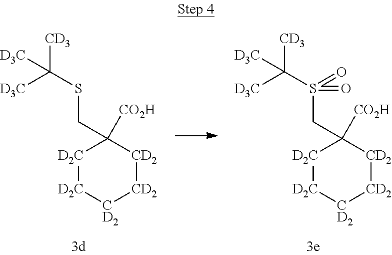

Oxone (25.0 g, 40.6 mmol) was suspended in water (35 mL) and was added into a solution of 3d (3.84 g, 15.4 mmol) in MeOH (50 mL). The solution was stirred at room temperature for overnight. The solid was removed by filtration and washed with MeOH (20 mL×3). The filtrate was concentrated. The residue was dissolved in EtOAc (250 mL) and washed with water (100 mL×2) and brine (100 mL). It was then dried over anhydrous Na₂SO₄, filtered, concentrated and dried under vacuum to give 3e (2.88 g, 66.5%).

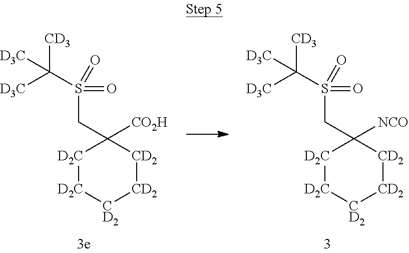

To a flamed-dried round bottom flask was added 3e (500 mg, 1.78 mmol) and anhydrous toluene (5 mL). Diphenylphosphoryl azide (0.158 mL, 0.734 mmol) and triethylamine (0.102 mL, 0.734 mmol) were added. The solution was stirred at 110° C. for 3.5 h. After cooling down, saturated NaHCO₃ (5 mL) was added. The aqueous layer was separated and extracted with toluene (2 ml). The toluene solution was combined and used without further purification.

### Synthesis of (S)-3-amino-N-cyclopropyl-6,6-dideutero-2-oxoheptanamide hydrochloride (4)

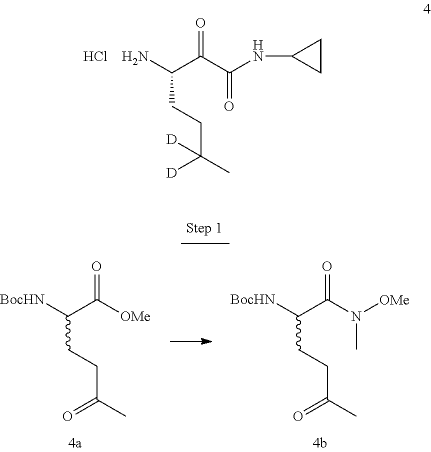

To a stirred solution of the ketone 4a (1.80 g, 6.9 mmol, prepared according to Chemistry Letters 1987, pp 2091-2094) in dioxane (20 ml) and water (7 mL) was added lithium hydroxide (0.183 g; 15.2 mmol) and the resulting mixture was stirred at room temperature overnight. The reaction was partitioned between CH₂Cl₂ and 1M aq. HCl. The aqueous phase was separated and further extracted with methylene chloride (X3). The combined organic layers were dried (MgSO₄), filtered, and concentrated to give the desired acid, used in the next step without purification.

To the crude acid in methylene chloride (20 ml) was added N,O-dimethylhydroxylamine:hydrochloride (0.817 g; 8.3 mmol), BOP reagent (3.69 g; 8.3 mmol) and triethylamine (3.49 ml; 25 mmol) and the resulting mixture was stirred at room temperature overnight. The reaction mixture was partitioned between EtOAc and 10% aq. HCl. The organic phase was separated, washed with sat aq. sodium bicarbonate, water, dried (MgSO₄) and the volatiles were removed under reduced pressure to give a residue which was purified by silica gel column chromatography to give the desired amide 4b (1.662 g) as a pale-yellow oil.

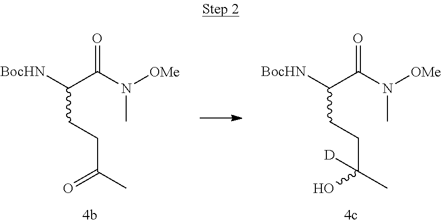

A stirred solution of the ketone 4b (1.66 g, 5.8 mmol) in dry MeOH (50 mL) was added sodium borodeuteride (0.364 g; 8.7 mmol) and the resulting mixture was stirred at room temperature for 2 h. Water was added and the methanol was removed under reduced pressure. The residue was partitioned CH₂Cl₂ and water. The aqueous phase was separated and further extracted with methylene chloride (X3). The combined organic layers were dried (MgSO₄), filtered, and concentrated to give a residue which was purified by silica gel column chromatography to give a diastereomeric mixture of alcohols (4c, 0.962 g) as a colorless oil.

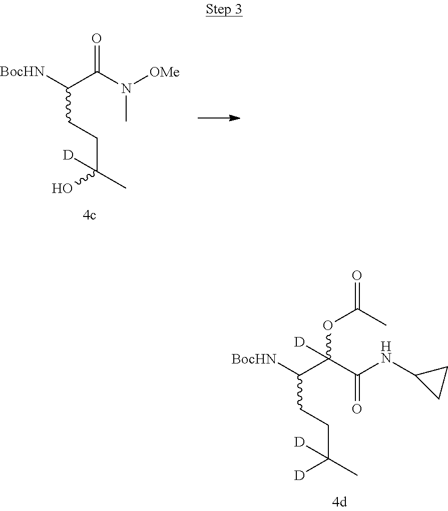

Triethylamine (0.46 ml; 3.3 mmol) was added to a mixture of the alcohols 4c (0.962 g; 3.3 mmol) tosyl chloride (0.757 g; 1.2 eq) and DMAP (0.403 g; 3.3 mmol) in dry CH₂Cl₂ (5 ml) while cooled in an ice bath. The resulting mixture was allowed to stir to room temperature overnight. The reaction mixture was partitioned between methylene chloride and 10% aq. HCl. The organic phase was separated, washed with sat. aq. sodium bicarbonate, dried (MgSO₄), filtered, and concentrated to give a residue which was used in the next step without purification.

Lithium aluminum deuteride (0.181 g; 4.1 mmol) was added to a stirred solution of the intermediate tosylate in anhydrous ether (30 ml), while cooled in an ice bath, under an atmosphere of nitrogen. The reaction is stirred for 0.5 h. and a solution of KHSO4 (0.787 g) in water (15 ml) was carefully added and portioned between EtOAc and 10% aq. HCl. The organic phase was separated, washed with sat. aq. sodium bicarbonate, water, dried (MgSO₄) and the volatiles removed under reduced pressure to give the crude aldehyde which was used without purification.

Cyclopropylisocyanide (0.266 g) in methylene chloride (5 ml) was added to the crude aldehyde (all material from previous step) and acetic acid (0.397 g; 2 eq.) was added dropwise. The resulting mixture was stirred at room temperature for 72 h. The volatiles were removed under reduced pressure and the residue was purified by silica gel chromatography to give the desired acetate 4d (0.368 g).

Representative procedure for the synthesis of final compound:

### Synthesis of Inhibitor XXI

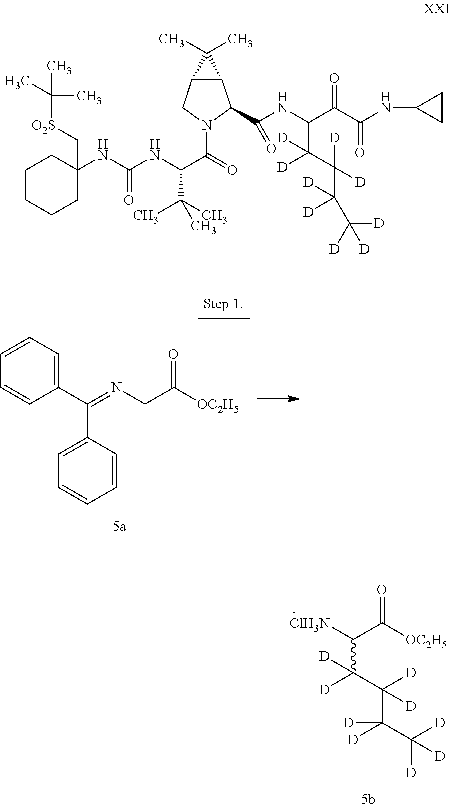

A stirred solution of ketimime 5a (7.0 g, Aldrich, 26 mmol) under N₂ in dry THF (50 mL) was cooled to −78° C. and treated with 1 M solution of LiHMDS (26 mL, 1 equiv.) in THF. The reaction mixture was stirred for 0.5 h and treated with D9-iodobutane (28 mL, Cambridge Isotope Laboratories Inc., 26 mmol). The reaction mixture was allowed to warm to room temperature overnight and treated aq. HCl (1 M, approx. 100 mL). The resulting solution was stirred at room temperature for 2 h and extracted with CH₂Cl₂ (X3). The aqueous layer was partitioned between sat. aq. sodium bicarbonate and methylene chloride. The aqueous phase was further extracted with methylene chloride (X3). The combined organic layers were dried (MgSO₄), filtered, and concentrated to give pure amine (5b, 3.09 g) as a colorless oil.

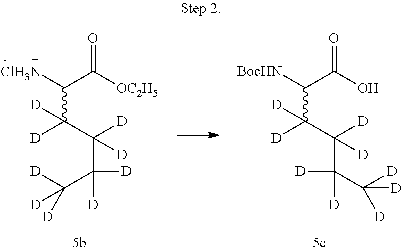

A solution of amine salt 5b (2.02 g, 12 mmol) at 0° C. in CH₂Cl₂ (25 mL) was treated with di-tert-butyldicarbonate (3.4 g, 15.6 mmol) and stirred at rt. for 12 h. After the completion of the reaction (TLC), the reaction mixture was concentrated in vacuo and the residue purified by silica gel chromatography to give 3.08 g of the intermediate ethyl ester which was dissolved in dioxane/H₂O (40 ml, 3:1) and treated with LiOH (0.522 g, 22 mmol) and stirred at room temperature overnight. The reaction mixture was partitioned between sat aq. sodium bicarbonate and CH₂Cl₂. The organic phase was separated and the aqueous layer further extracted with CH₂Cl₂. The combined organic layers were dried (MgSO₄), filtered, and concentrated in vacuo to yield 5c (3.078 g) which was used in next step without any further purification.

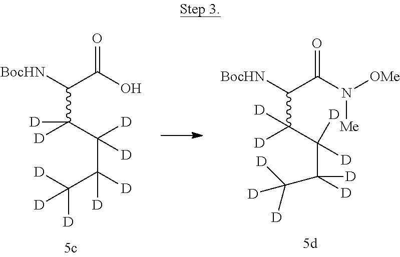

A solution of acid 5c (3.078 g, 12.8 mmol) in CH₂Cl₂ (250 mL) was treated with BOP reagent (6.81 g, 15.4 mmol), triethylamine (6.43 mL), N,O-dimethylhydroxylamine hydrochloride (1.50 g, 15.4 mmol) and stirred overnight at rt. The reaction mixture was diluted with 1 N aq. HCl, and the layers were separated and the aqueous layer was extracted with CH₂Cl₂ (3×300 ml). The combined organic layers were dried (MgSO₄), filtered, concentrated in vacuo and purified by chromatography (SiO₂, EtOAc/Hex 1:5) to yield the amide 5d (2.62 g) as a white solid.

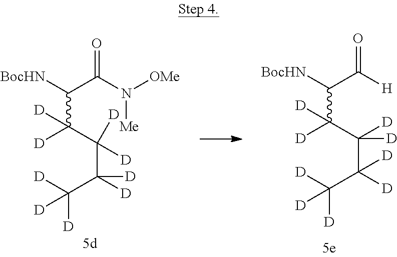

A solution of amide 5d (1.00 g, 3.5 mmol) in dry diethyl ether (35 mL) was treated LiAlH₄ (0.166 g; 17.5 mmol) at 0° C. The reaction mixture was stirred at room temperature for 1 h and carefully quenched at 0° C. with a solution of KHSO₄ (10% aq.) and stirred for 0.5 h. The reaction mixture was diluted with aq. HCl (1 M, 150 mL) and extracted with CH₂Cl₂ (3×200 mL), The combined organic layers were washed with aq. HCl (1 M), saturated NaHCO₃, brine, and dried (MgSO₄). The mixture was filtered and concentrated in vacuo to yield 5e (0.282 g).

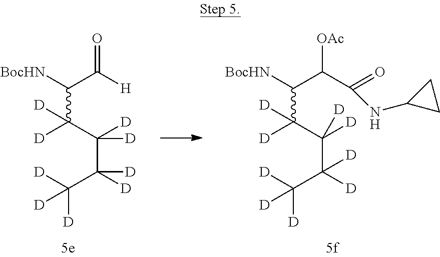

A solution of the aldehyde 5e (0.782 g, 3.5 mmol) in CH₂Cl₂ (40 mL), was treated with cyclopropylisonitrile (0.261 g mL, 3.8 mmol), and acetic acid (0.42 g g, 7 mmol) and stirred at room temperature for 2 weeks. The reaction mixture was concentrated in vacuo and purified by chromatography (SiO₂, EtOAc/Hex 2:3) to yield 5f (0.93 g) as a white solid, a mixture of diastereomers.

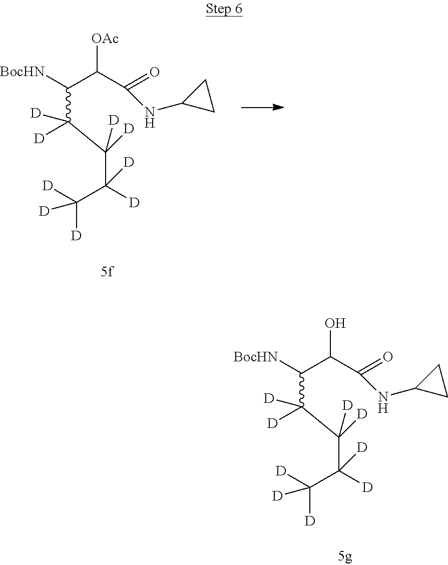

Potassium carbonate (50 mgs) was added to a stirred solution of the acetate 5f (0.876 g mL) in methanol (10 ml). The reaction mixture was then stirred at room temperature for 1 h. and partitioned between EtOAc and sat aq. sodium bicarbonate. The organic phase was separated, dried (MgSO₄) and concentrated to give the alcohols 5 g 1 g (0.793 g) as a white solid.

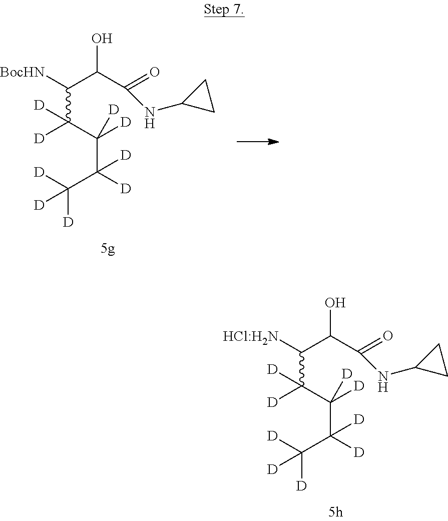

A solution of HCl in dioxane (10 mL of 4M) to the carbonate 5 g (0.79 g) and Boc₂O (45.7 g, 209 mmol) and left at room temperature for 3 h. the reaction mixture was concentrated in vacuo to give the hydrochloride salt 5 h (0.596 g).

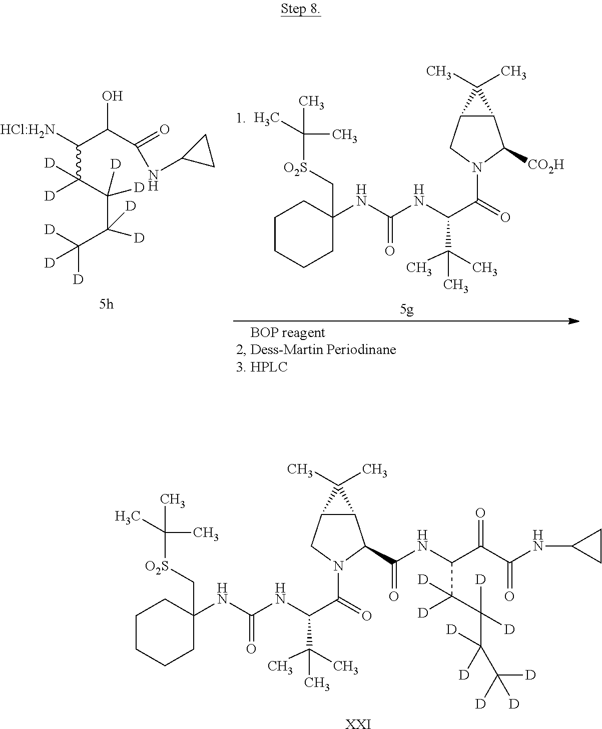

To a mixture of the hydrochloride salt 5 h (0.2 g; 0.8 mmol) and the carboxylic acid 5 g (0.429 g; 0.8 mmol; prepn. WO2005087731) in CH₂Cl₂ (10 mL) was added BOP reagent followed by triethylamine (0.41 ml; 2.9 mmol) and the resulting mixture was stirred at room temperature overnight. The reaction mixture was partitioned between EtOAc and 1M HCl (aq.). The organic phase was separated, washed with sat. aq. sodium bicarbonate, water, dried (MgSO₄), and. the solvents were removed under vacuo. to give the crude intermediate α-hydroxyamide, which was used in the next step without purification.

All of the crude product from the previous step was dissolved in. CH₂Cl₂ (10 ml) and Dess-Martin periodinane (0.69 g; 1.6 mmol) was added and the resulting mixture was stirred at room temperature for a period of 2 h. The reaction mixture was partitioned between EtOAc and 10% aq. sodium thiosulfate. The organic phase was separated, washed with sat aq. sodium bicarbonate, water, dried (MgSO₄) and concentrated in vacuo. The residue was purified by silica gel chromatography using EtOAc; hexanes (1:1) as eluent to give a mixture of diastereomers, which were subsequently separated by HPLC as shown below.

### Separation of Diastereomers of Compound XXI

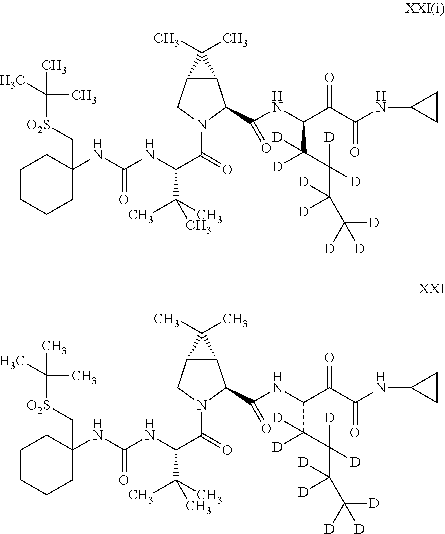

### Preparative HPLC Condition for Separation

### COLUMN USED: NORMAL PHASE YMC DIOL-NP COLUMN

### SOLVENT A: Hexanes

SOLVENT B: To make 4 L of solvent (1.7 L Isopropanol, +300 mL of
CH₃CN+2 L of CH₂Cl₂)

### HPLC CONDITIONS: 8% of Solvent B/88% of Solvent A

FLOW: 25 mL/min
Procedure: 0.05 g of compound XXI was dissolved in hexanes and CH₂Cl₂ and injected into the column. It was eluted with 20 mL/min and two peaks were independently collected and concentrated. The solid residue was further dried in high vacuum. Gave the desired polar isomer XXI (0.0196 g)

### Synthesis of Inhibitors XXII.

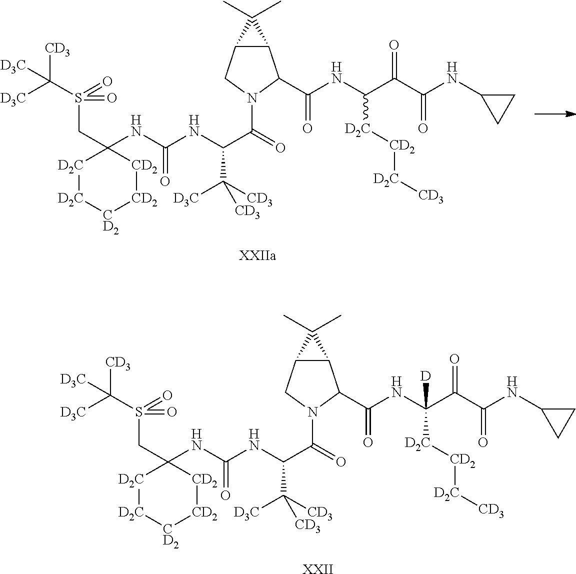

To a flamed-dried round bottom flask was added XXIIa (162 mg), anhydrous CH₂Cl₂ (3 ml), and D₂O (0.5 ml). Triethyl amine (3 drops) was then added. The solution was stirred at room temperature for overnight under N₂. CD₃CD₂OD (5 drops) was added and the solution was stirred at room temperature for 5 min. The organic layer was separated. The aqueous layer was extracted with CH₂Cl₂ (1 mL) once. The organic layers were combined, concentrated, and dried under vacuum for 2 h. The product was separated by HPLC using conditions previously described to give XXII (72.5 mg, 44.8%).

| Entry | Structure | ¹H NMR and MS |
|---|---|---|
| XXIII | 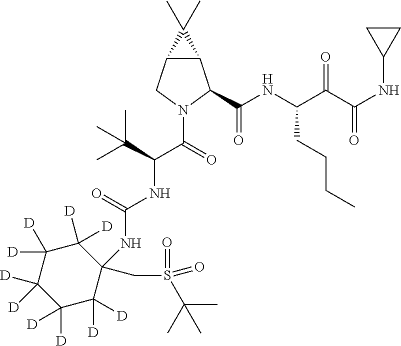 | — |
| XXI | 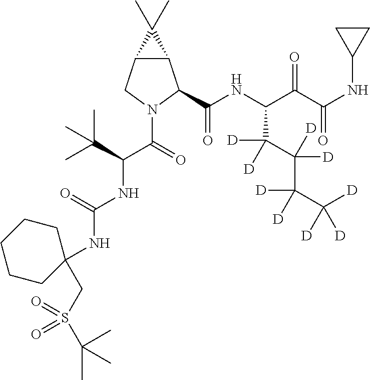 | ¹H NMR (CDCl₃, 500 MHz): δ, 7.58 (d, 1H, J = 7.0 Hz), 7.18 (br.s, 1H), 5.54 (d, 1H, J = 7.0 Hz), 5.38 (d, 1H, J = 9.8 Hz), 4.92 (br.s, 1H), 4.57 (s, 1H), 4.43 (d, 1H, J = 10 Hz), 4.08 (d, 1H, J = 13.5 Hz), 4.01 (d, 1H, J = 10.5 Hz), 3.78-3.85 (m, 1H), 2.85 (d, 1H, J = 13.5 Hz), 2.77-2.84 (m, 1H), 2.45-2.55 (m, 1H), 2.16-2.24 (m, 1H), 1.67-1.77 (m, 2H), 1.34 (s, 9H), 1.20-1.59 (m, 8H), 1.01 (s, 3H), 0.95 (s, 9H), 0.85 (s, 3H), 0.79-0.89 (m, 2H) and 0.60-0.68 (m, 2H). ¹³C NMR (CDCl₃, 125 MHζ): δ, 197.19, 171.12, 170.38, 162.00, 156.78, 59.16, 59.00, 56.34, 53.50, 53.38, 50.56, 47.39, 34.38 (2xC), 34.15, 30.71, 26.87, 26.25 (3xC), 26.13, 24.92, 22.47 (3xC), 22.45, 20.59, 20.54, 18.43, 12.55, 5.38 and 5.35 . . . [M + 1]⁺ 717.4 . . . |
| XXV | 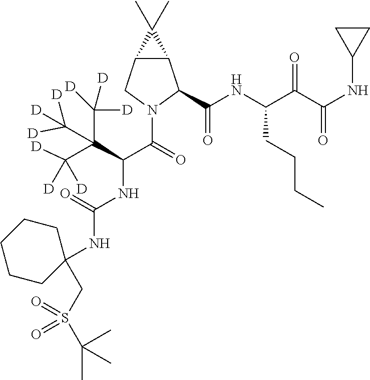 | ¹H NMR (d₆-DMSO, 500 MHz): δ, 8.72 (d, 1H, J = 5.9 Hz), 8.34 (d, 1H, J = 6.6 Hz), 6.18 (d, 1H, J = 9.5 Hz), 6.03 (s, 1H), 4.95-4.90 (m, 1H), 4.27 (s, 1H), 4.14 (d, 1H, J = 9.5 Hz), 3.87 (d, 1H, J = 10.3 Hz), 3.70 (dd, 1H, J = 5.1 & 3.7 Hz), 3.68 (d, 1H, J = 13.2 Hz), 3.28 (d, 1H, J = 14 Hz), 2.76-2.69 (m, 1H), 2.23-2.14 (m, 2H), 1.75-1.67 (m, 1H), 1.75-0.80 (m, 15H) 1.21 (s, 9H), 0.98 (s, 3H), 0.84 (t, 3H, J = 7.3 Hz), 0.80 (s, 3H), 0.66- 0.61 (m, 2H), 0.57-0.53 (m, 2H). LCMS calcd. for C₃₆H₂₂D₉N₅O₇S [M + 1]⁺ 717; observed: 717 |
| XXVI | 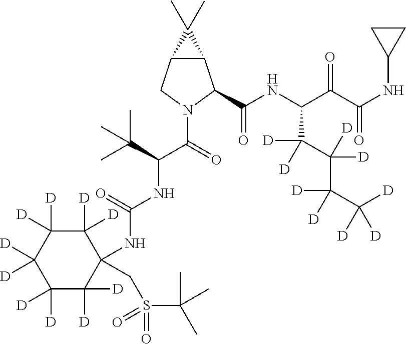 | ¹H NMR (CDCl3, 500 MHz): δ, 7.39 (d, 1H, J = 6.5 Hz), 7.02 (d, 1H, J = 3.5 Hz), 5.33 (d, 1H, J = 7.0 Hz), 5.13 (d, 1H, J = 10.5 Hz), 4.64 (s, 1H), 4.51 (s, 1H), 4.44 (d, 1H, J = 9.5 Hz), 4.10 (d, 1H, J = 14 Hz), 4.08 (d, 1H, J = 10.5 Hz), 3.81 (dd, 1H, J = 11 & 5.5 Hz), 2.90 (d, 1H, J = 13.5 Hz), 2.81 (m, 1H), 1.67 (d, 1H, J = 7.5 Hz), 1.49 (dd, 1H, J = 7.5 & 5 Hz), 1.37 (s, 9H), 1.04 (s, 3H), 0.99 (s, 9H), 0.90 (t, 3H, J = 7 Hz), 0.86 (s, 3H), 0.64 (m, 2H). LCMS calcd. for C₃₆H₄₂D₁₉N₅O₇S [M + 1]⁺ 727.5; observed: 727.4. |
| XXVII | 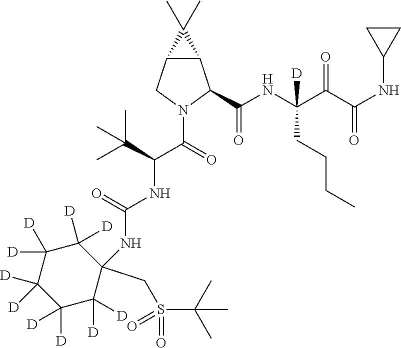 | ¹H NMR (CDCl3, 500 MHz): δ, 7.44 (s, 1H), 7.06 (d, 1H, J = 3.0 Hz), 5.19 (d, 1H, J = 10.0 Hz), 4.70 (s, 1H), 4.53 (s, 1H), 4.44 (d, 1H, J = 9.5 Hz), 4.11 (d, 1H, J = 13.5 Hz), 4.07 (d, 1H, J = 11 Hz), 3.81 (dd, 1H, J = 10 & 5 Hz), 2.89 (d, 1H, J = 14 Hz), 2.82 (m, 1H), 1.96 (m, 1H), 1.64 (m, 1H), 1.50 (dd, 1H, J = 7.5 & 5 Hz), 1.37 (s, 9H), 1.04 (s, 3H), 0.99 (s, 9H), 0.90 (t, 3H, J = 7 Hz), 0.86 (s, 3H), 0.64 (m, 2H). LCMS calcd. for C₃₆H₅₀D₁₁N₅O₇S [M + 1]⁺ 719.5; observed: 719.4. |
| XXVIII | 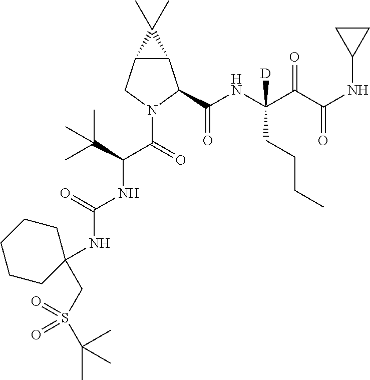 | ¹H NMR (CDCl₃; 400 MHz): δ 7.45 (1H, s), 7.07 (1H, d, J = 4.27 Hz), 5.25 (1H, d, J = 10.37 Hz), 4.75 (1H, s), 4.51 (1H, s), 4.42 (1H, d, J = 9.76 Hz), 4.08 (1H, d, J = 13.42 Hz), 4.04 (1H, d, J = 10.98 Hz), 3.79 (1H, dd, J = 5.49,10.37 Hz), 2.88 (1H, d, J = 13.42 Hz), 2.80 (1H, m), 2.48 (1H, m), 2.23 (1H, d, J = 12.20 Hz), 1.94 (1H, m), 1.73 (1H, m), 1.34 (9H, s), 1.20-1.68 (14H, m), 1.01 (3H, s), 0.96 (9H, s), 0.88 (3H, t, J = 7.32 Hz), 0.86 (2H, m), 0.84 (3H, s), 0.63 (2H, m) |
| XXIX | 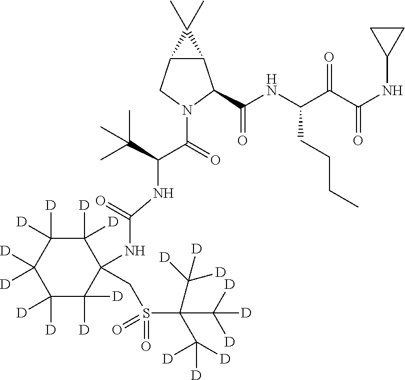 | ¹H NMR (CDCl3, 500 MHz): δ, 7.46 (d, 1H, J = 6.5 Hz),7.09 (m, 1H), 5.37 (m, 1H), 5.25(s, 1H), 4.78 (s, 1H), 4.44 (d, 1H, J = 9.5 Hz), 4.08 (d, 1H, J = 14 Hz), 4.07 (d, 1H, J = 11 Hz), 3.82 (dd, 1H, J = 11 & 5.5 Hz), 2.89 (d, 1H, J = 14 Hz), 2.82 (m, 1H), 1.98 (m, 1H), 1.62 (m, 2H), 1.50 (dd, 1H, J = 7.5 & 5 Hz), 1.36 (m, 4H), 1.03 (s, 3H), 0.98 (s, 9H), 0.90 (t, 3H, J = 7 Hz), 0.86 (s, 3H), 0.64 (m, 2H). LCMS calcd. for C₃₆H₄₂D₁₉N₅O₇S [M + 1]⁺ 727.6; observed: 727.4. |
| XXX | 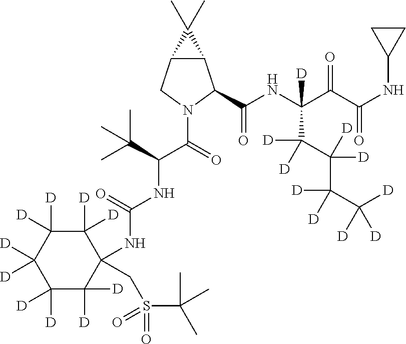 | ¹H NMR (CDCl3, 500 MHz): δ, 7.53 (s, 1H), 7.06 (d, 1H, J = 4.0 Hz), 5.39 (m 1H), 4.88 (s, 1H), 4.56 (s, 1H), 4.46 (d, 1H, J = 10 Hz), 4.10 (d, 1H, J = 14 Hz),4.05 (d, 1H, J = 10.5 Hz), 3.83 (dd, 1H, J = 10.5 & 5 Hz), 2.89 (d, 1H, J = 14 Hz), 2.82 (m, 1H), 1.57 (d, 1H, J = 7.5 Hz), 1.50 (dd, 1H, J = 8 & 5 Hz), 1.37 (s, 9 H), 1.03 (s, 3H), 0.98 (s, 9 H), 0.86 (s, 3 H), 0.64 (m, 2H). LCMS calcd. for C₃₆H₄₁D₂₀N₅O₇S [M + 1]⁺ 728.6; observed: 728.4. |
| XXXI | 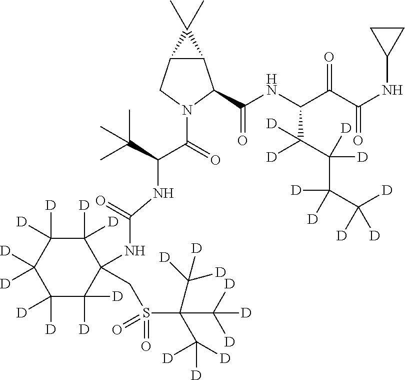 | ¹H NMR (CDCl3, 500 MHz): δ, 7.42 (d, 1H, J = 7.0 Hz), 7.06 (d, 1H, J = 3.5 Hz), 5.34 (d, 1H, J = 7.5 Hz), 5.16 (d, 1H, J = 10.5 Hz), 4.67 (s, 1H), 4.52 (s, 1H),4.44 (d, 1H, J = 10.5 Hz), 4.10 (d, 1H, J = 13.5 Hz), 4.08 (d, 1H, J = 11.5 Hz), 3.81 (dd, 1H, J = 11 & 5.5 Hz), 2.88 (d, 1H, J = 13.5 Hz), 2.81 (m, 1H), 1.65 (d, 1H, J = 7.5 Hz), 1.49 (dd, 1H, J= 7.5 & 5 Hz), 1.04 (s, 3H), 0.98 (s, 9 H), 0.84 (s, 3 H), 0.64 (m, 2H). LCMS calcd. for C₃₆H₃₃D₂₈N₅O₇S [M + 1]⁺ 736.6; observed: 736.4. |
| XXXII | 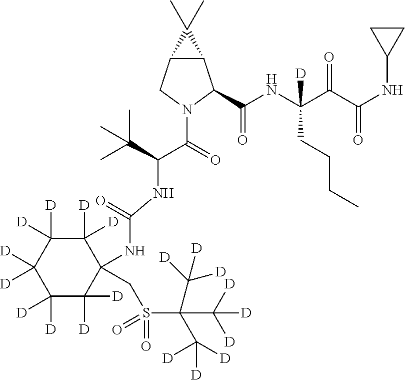 | ¹H NMR (CDCl3, 500 MHz): δ, 7.48 (s, 1H),7.1 (d, 1H, J = 3 Hz), 5.27 (d, 1H, J = 9.5 Hz), 4.76 (s, 1H), 4.54 (s, 1H), 4.45 (d, 1H, J = 10 Hz), 4.10 (d, 1H, J = 13 Hz), 4.07 (d, 1H, J = 10.5 Hz), 3.82 (dd, 1H, J = 10.5 & 5.5 Hz), 2.88 (d, 1H, J = 13.5 Hz), 2.82 (m, 1H), 1.98 (m, 1H), 1.66 (m, 2H), 1.62 (d, 1H, J = 7.5 Hz), 1.50 (dd, 1H, J = 7.5 & 5 Hz), 1.34 (m, 4H), 1.03 (s, 3H), 0.98 (s, 9H), 0.90 (t, 3H, J = 7 Hz), 0.86 (s, 3 H), 0.64 (m, 2H). LCMS calcd. for C₃₆H₄₁D₂₀N₅O₇S [M + 1]⁺ 728.6; observed: 728.5. |
| XXXIII | 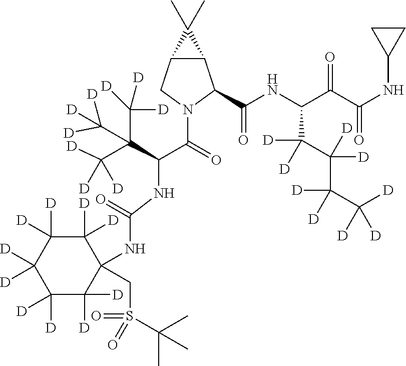 | ¹H NMR (CDCl₃, 500 MHz): δ, 7.50 (d, 1H, J = 6.7 Hz), 7.11 (br.s, 1H), 5.31-5.40 (m, 2H), 4.85 (br.s, 1H), 4.52 (s, 1H), 4.46 (d, 1H, J = 9.9 Hz), 3.98-4.08 (m, 2H), 3.78- 3.83 (m, 1H), 2.86 (d, 1H, J = 13.6 Hz), 2.77-2.82 (m, 1H), 1.54-1.59 (m, 1H), 1.42-1.49 (m, 1H), 1.37 (s, 9H), 1.00 (s, 3H), 0.84 (s, 3H), 0.78-0.90 (m, 2H) and 0.57- 0.67(m, 2H). ¹³C NMR (CDCl₃, 125 MHz): δ, 197.25, 172.59, 170.55, 160.34, 157.16, 60.27, 59.67, 57.58, 54.26, 53.88, 50.41, 48.41, 34.34 , 29.25, 27.36, 26.40, 23.01 (3xC), 22.52, 18.62, 12.59, 6.36 and 6.35 |
| XXII | 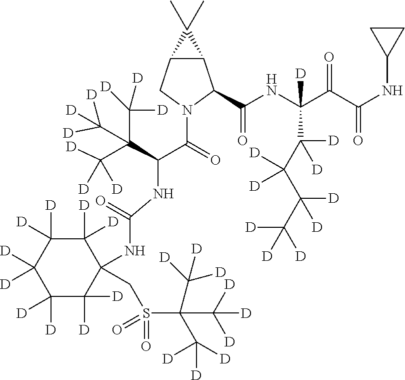 | ¹H NMR (CDCl3, 500 MHz): δ, 7.41 (s, 1H), 7.05 (d, 1H, J = 3.5 Hz), 5.22 (d, 1H, J = 9.5 Hz), 4.73 (s, 1H), 4.52 (s, 1H), 4.43 (d, 1H, J = 9.5 Hz), 4.08 (s, 1H), 4.06 (d, 1H, J = 4.5 Hz), 3.81 (dd, 1H, J = 10 & 5 Hz), 2.91 (d, 1H, J = 13.5 Hz), 2.81 (m, 1H), 1.49 (dd, 1H, J = 7.5 & 5 Hz), 1.04 (s, 3H), 0.84 (s, 3H), 0.64 (m, 2H). LCMS calcd. for C₃₆H₂₃D₃₈N₅O₇S [M + 1]⁺ 746.7; observed: 746.6. |
| IV | 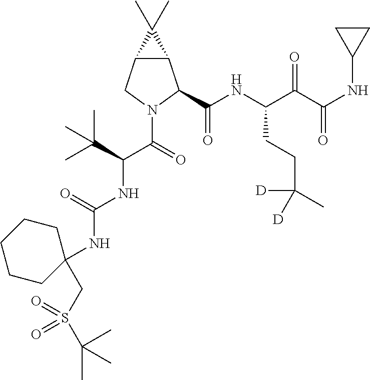 | ¹H NMR (CDCl₃, 500 MHz): δ, 7.55 (d, 1H, J = 7.2 Hz), 7.15 (d, 1H, J = 3.6 Hz), 5.34-5.42 (m, 2H), 4.88 (br.s, 1H), 4.53 (s, 1H),4.44(d, 1H, J = 9.9 Hz), 4.07 (d, 1H, J = 13.6 Hz), 4.00 (d, 1H, J = 10.5 Hz), 3.78- 3.82 (m, 1H), 2.85 (d, 1H, J = 13.6 Hz), 2.78-2.83 (m, 1H), 2.45-2.52 (m, 1H), 2.15-2.23 (m, 1H), 1.90- 1.99 (m, 1H), 1.65-1.75 (m, 2H), 1.35 (s, 9H), 1.20-1.6 (m, 11H), 1.00 (s, 3H), 0.95 (s, 9H), 0.85 (s, 3H), 0.82 (s, 3H), 0.79-0.84 (m, 2H) and 0.59-0.65 (m, 2H). ¹³C NMR (CDCl₃, 125 MHz): δ, 197.39, 172.53, 170.54, 160.31, 157.16, 60.29, 59.62, 57.64, 54.54, 53.96, 50.48, 48.43, 35.91, 35.53, 35.08, 30.88, 29.28, 27.63, 27.34 26.42, 26.39 (3xC), 25.37, 23.02 (3xC), 22.55, 21.17, 21.04, 18.59, 13.50, 12.59, 6.37 and 6.33. |

The present invention relates to and discloses novel HCV protease inhibitors. This utility can be manifested in their ability to inhibit the HCV NS2/NS4a serine protease. A general procedure for such demonstration is illustrated by the following in vitro assay.

### Assay for HCV Protease Inhibitory Activity:

Spectrophotometric Assay Spectrophotometric assay for the HCV serine protease can be performed on the inventive compounds by following the procedure described by R. Zhang et al, Analytical Biochemistry, 270 (1999) 268-275, the disclosure of which is incorporated herein by reference. The assay based on the proteolysis of chromogenic ester substrates is suitable for the continuous monitoring of HCV NS3 protease activity. The substrates are derived from the P side of the NS5A-NS5B junction sequence (Ac-DTEDVVX(Nva), where X=A or P) whose C-terminal carboxyl groups are esterified with one of four different chromophoric alcohols (3- or 4-nitrophenol, 7-hydroxy-4-methyl-coumarin, or 4-phenylazophenol). Illustrated below are the synthesis, characterization and application of these novel spectrophotometric ester substrates to high throughput screening and detailed kinetic evaluation of HCV NS3 protease inhibitors.

### Materials and Methods:

Materials: Chemical reagents for assay related buffers are obtained from Sigma Chemical Company (St. Louis, Mo.). Reagents for peptide synthesis were from Aldrich Chemicals, Novabiochem (San Diego, Calif.), Applied Biosystems (Foster City, Calif.) and Perseptive Biosystems (Framingham, Mass.). Peptides are synthesized manually or on an automated ABI model 431A synthesizer (from Applied Biosystems). UVNIS Spectrometer model LAMBDA 12 was from Perkin Elmer (Norwalk, Conn.) and 96-well UV plates were obtained from Corning (Corning, N.Y.). The prewarming block can be from USA Scientific (Ocala, Fla.) and the 96-well plate vortexer is from Labline Instruments (Melrose Park, Ill.). A Spectramax Plus microtiter plate reader with monochrometer is obtained from Molecular Devices (Sunnyvale, Calif.).

Enzyme Preparation Recombinant heterodimeric HCV NS3/NS4A protease (strain 1a) is prepared by using the procedures published previously (D. L. Sali et al, Biochemistry, 37 (1998) 3392-3401). Protein concentrations are determined by the Biorad dye method using recombinant HCV protease standards previously quantified by amino acid analysis. Prior to assay initiation, the enzyme storage buffer (50 mM sodium phosphate pH 8.0, 300 mM NaCl, 10% glycerol, 0.05% lauryl maltoside and 10 mM DTT) is exchanged for the assay buffer (25 mM MOPS pH 6.5, 300 mM NaCl, 10% glycerol, 0.05% lauryl maltoside, 5 μM EDTA and 5 μM DTT) utilizing a Biorad Bio-Spin P-6 prepacked column.
Substrate Synthesis and Purification: The synthesis of the substrates is done as reported by R. Zhang et al, (ibid.) and is initiated by anchoring Fmoc-Nva-OH to 2-chlorotrityl chloride resin using a standard protocol (K. Barlos et al, Int. J. Pept. Protein Res., 37 (1991), 513-520). The peptides are subsequently assembled, using Fmoc chemistry, either manually or on an automatic ABI model 431 peptide synthesizer. The N-acetylated and fully protected peptide fragments are cleaved from the resin either by 10% acetic acid (HOAc) and 10% trifluoroethanol (TFE) in dichloromethane (DCM) for 30 min, or by 2% trifluoroacetic acid (TFA) in DCM for 10 min. The combined filtrate and DCM wash is evaporated azeotropically (or repeatedly extracted by aqueous Na₂CO₃ solution) to remove the acid used in cleavage. The DCM phase is dried over Na₂SO₄ and evaporated.

The ester substrates are assembled using standard acid-alcohol coupling procedures (K. Holmber et al, Acta Chem. Scand., B33 (1979) 410-412). Peptide fragments are dissolved in anhydrous pyridine (30-60 mg/ml) to which 10 molar equivalents of chromophore and a catalytic amount (0.1 eq.) of para-toluenesulfonic acid (pTSA) were added. Dicyclohexylcarbodiimide (DCC, 3 eq.) is added to initiate the coupling reactions. Product formation is monitored by HPLC and can be found to be complete following 12-72 hour reaction at room temperature. Pyridine solvent is evaporated under vacuum and further removed by azeotropic evaporation with toluene. The peptide ester is deprotected with 95% TFA in. DCM for two hours and extracted three times with anhydrous ethyl ether to remove excess chromophore. The deprotected substrate is purified by reversed phase HPLC on a C3 or C8 column with a 30% to 60% acetonitrile gradient (using six column volumes). The overall yield following HPLC purification can be approximately 20-30%. The molecular mass can be confirmed by electrospray ionization mass spectroscopy. The substrates are stored in dry powder form under desiccation.

Spectra of Substrates and Products: Spectra of substrates and the corresponding chromophore products are obtained in the pH 6.5 assay buffer. Extinction coefficients are determined at the optimal off-peak wavelength in 1-cm cuvettes (340 nm for 3-Np and HMC, 370 nm for PAP and 400 nm for 4-Np) using multiple dilutions. The optimal off-peak wavelength is defined as that wavelength yielding the maximum fractional difference in absorbance between substrate and product (product OD−substrate OD)/substrate OD).
Protease Assay: HCV protease assays are performed at 30° C. using a 200 μl reaction mix in a 96-well microtiter plate. Assay buffer conditions (25 mM MOPS pH 6.5, 300 mM NaCl, 10% glycerol, 0.05% lauryl maltoside, 5 μM EDTA and 5 μM DTT) are optimized for the NS3/NS4A heterodimer (D. L. Sali et al, ibid.)). Typically, 150 μl mixtures of buffer, substrate and inhibitor are placed in wells (final concentration of DMSO≦4% v/v) and allowed to preincubate at 30° C. for approximately 3 minutes. Fifty pls of prewarmed protease (12 nM, 30° C.) in assay buffer, is then used to initiate the reaction (final volume 200 μl). The plates are monitored over the length of the assay (60 minutes) for change in absorbance at the appropriate wavelength (340 nm for 3-Np and HMC, 370 nm for PAP, and 400 nm for 4-Np) using a Spectromax Plus microtiter plate reader equipped with a monochrometer (acceptable results can be obtained with plate readers that utilize cutoff filters). Proteolytic cleavage of the ester linkage between the Nva and the chromophore is monitored at the appropriate wavelength against a no enzyme blank as a control for non-enzymatic hydrolysis. The evaluation of substrate kinetic parameters is performed over a 30-fold substrate concentration range (˜6-200 μM). Initial velocities are determined using linear regression and kinetic constants are obtained by fitting the data to the Michaelis-Menten equation using non-linear regression analysis (Mac Curve Fit 1.1, K. Raner). Turnover numbers (k_{ca}t) are calculated assuming the enzyme is fully active.
Evaluation of inhibitors and Lnactivators: The inhibition constants (Kᵢ) for the competitive inhibitors Ac-D-(D-Gla)-L-1-(Cha)-C—OH (27), Ac-DTEDVVA(Nva)-OH and Ac-DTEDVVP(Nva)-OH are determined experimentally at fixed concentrations of enzyme and substrate by plotting vₒ/vᵢ vs. inhibitor concentration ([I]ₒ) according to the rearranged Michaelis-Menten equation for competitive inhibition kinetics: vₒ/vᵢ=+[I]ₒ/(Kᵢ (1+[S]ₒ/Kₘ)), where vₒ is the uninhibited initial velocity, vᵢ is the initial velocity in the presence of inhibitor at any given inhibitor concentration ([I]ₒ) and [S]ₒ is the substrate concentration used. The resulting data are fitted using linear regression and the resulting slope, 1/(Kᵢ(1+[S]ₒ/Kₘ), is used to calculate the Kᵢ value,

1. A compound exhibiting HCV protease inhibitory activity, or enantiomers, stereoisomers, rotamers, tautomers, and racemates of said compound, or a pharmaceutically acceptable salt of said compound, said compound being selected from the compounds of structures listed below:

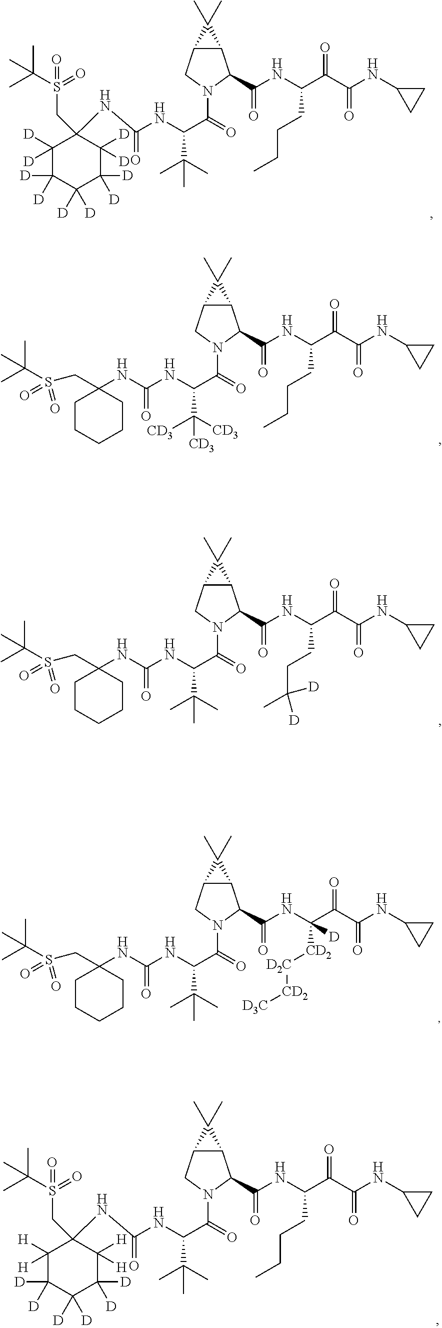

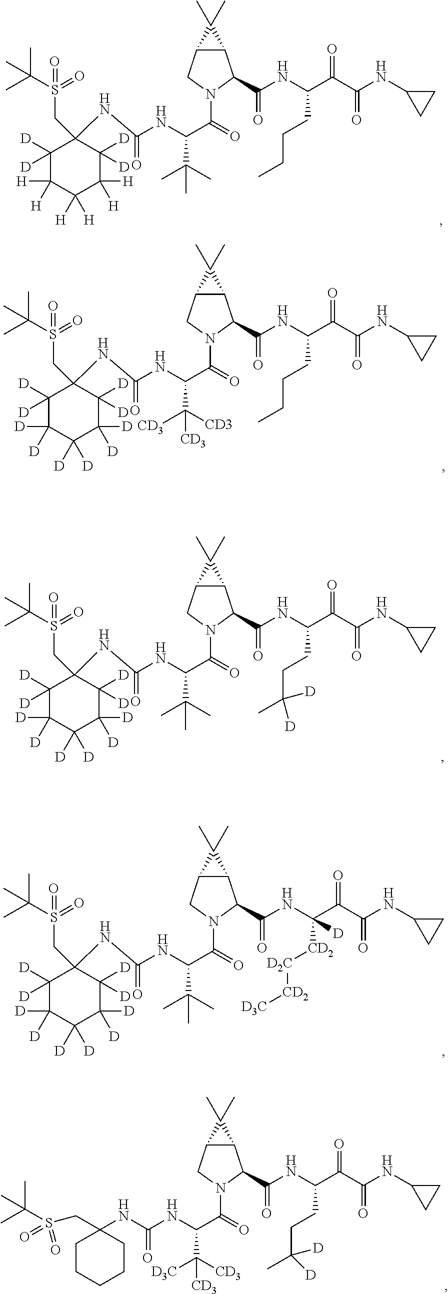

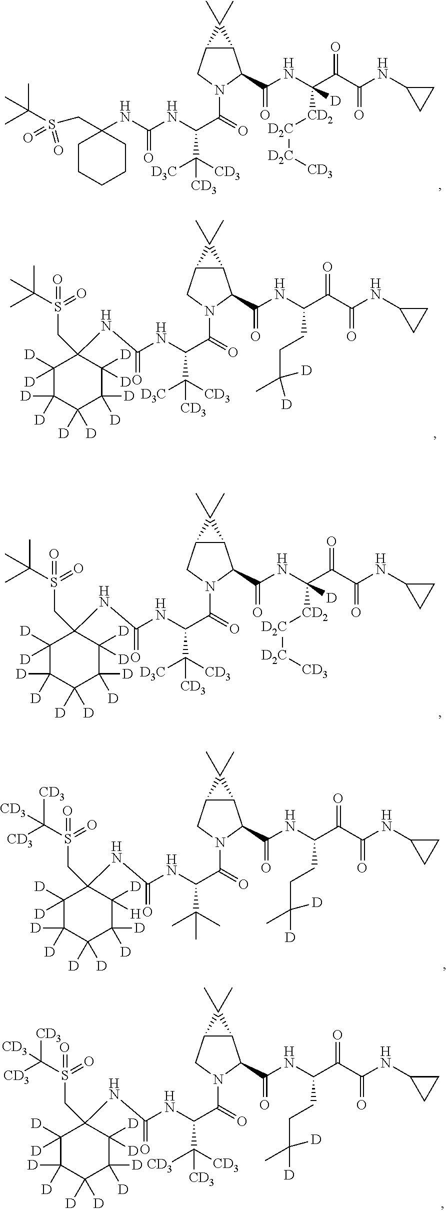

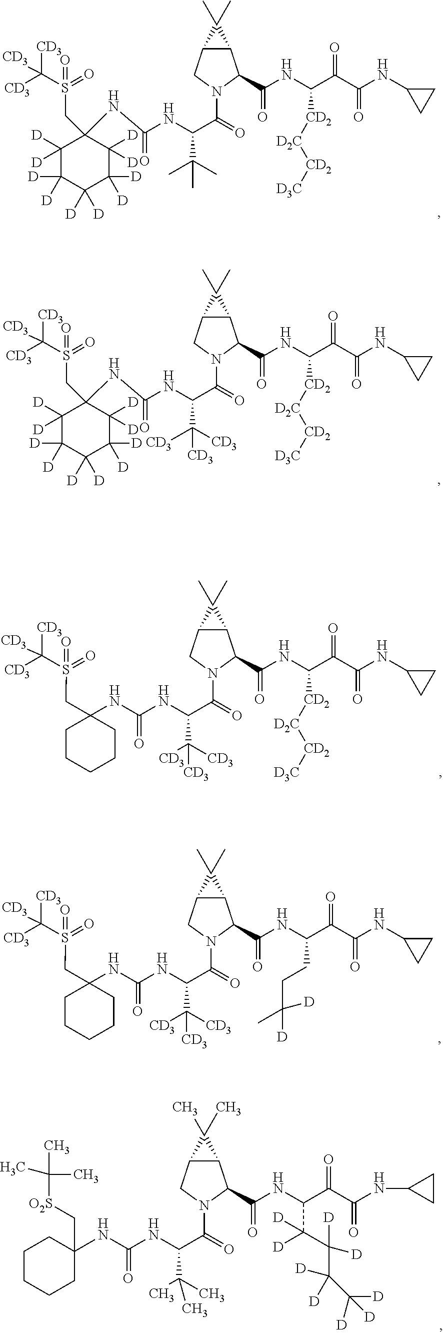

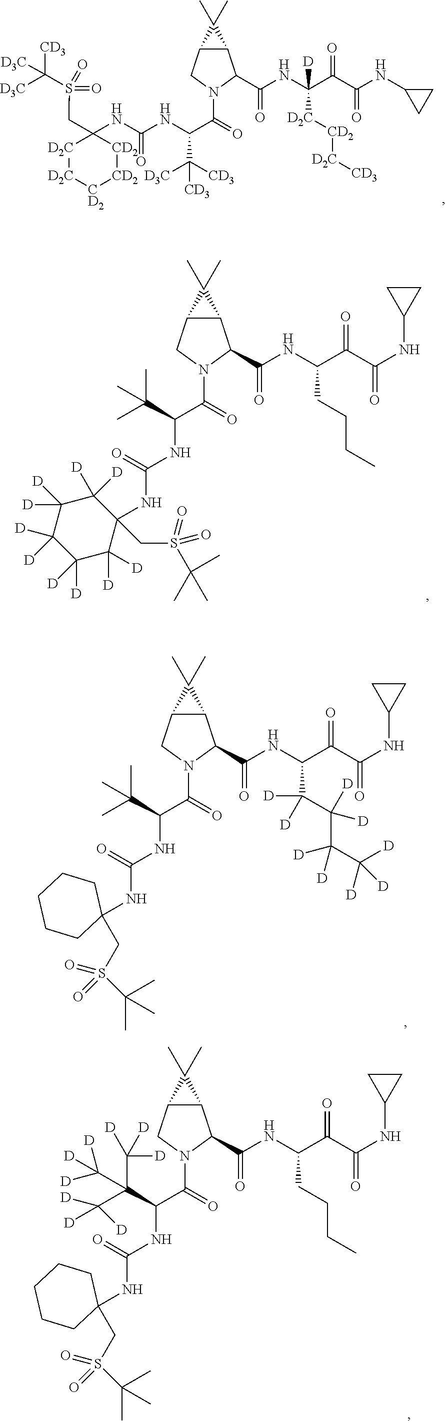

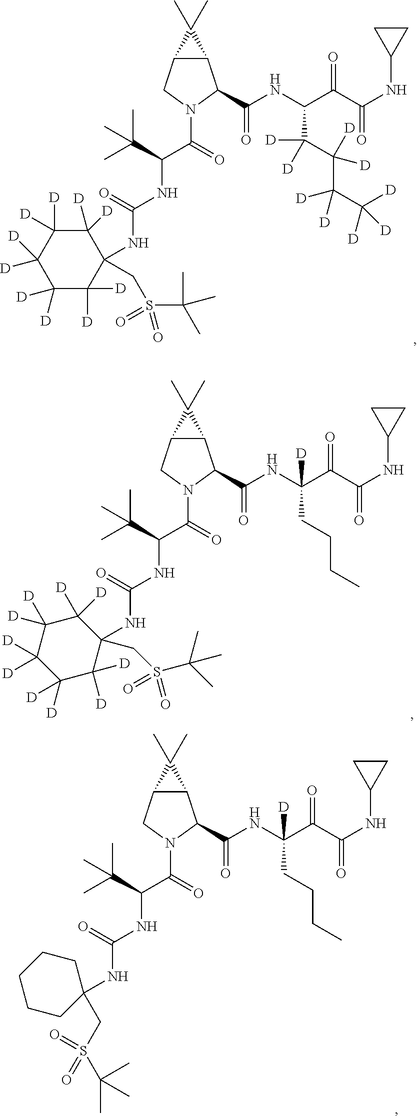

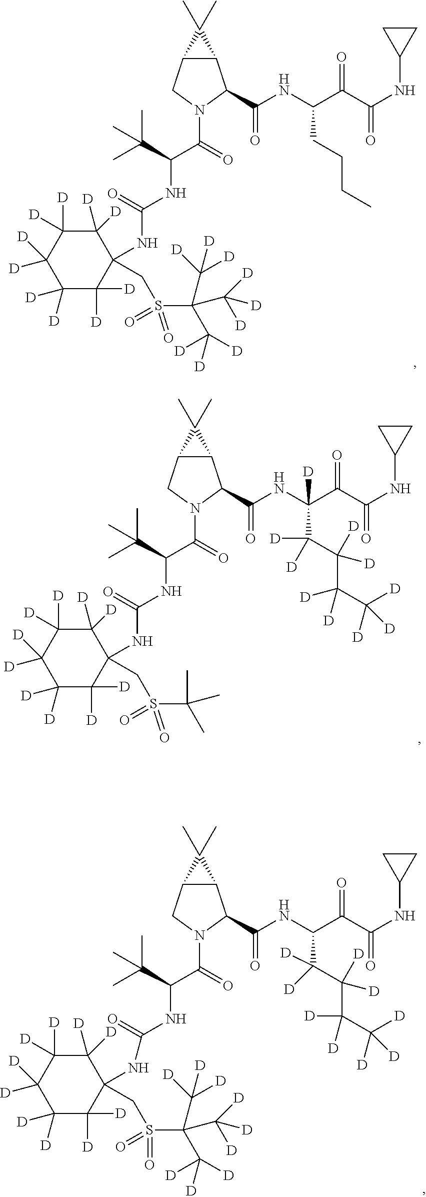

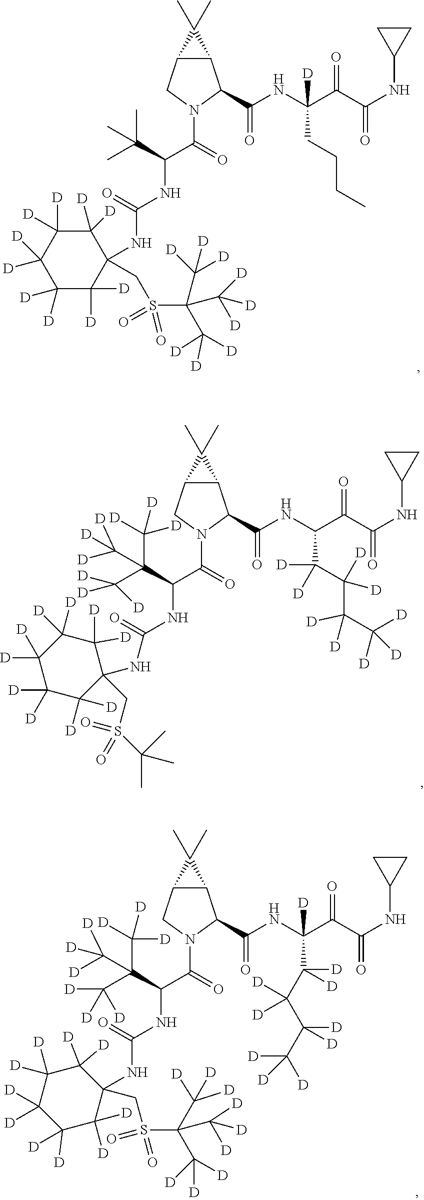

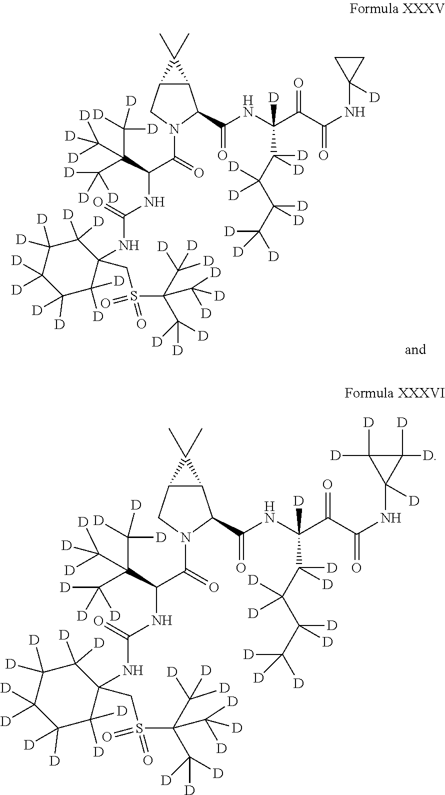

2. A pharmaceutical composition comprising as an active ingredient at least one compound of claim 1, or pharmaceutically acceptable salt thereof, and a pharmaceutically acceptable carrier.

3. The pharmaceutical composition of claim 2, additionally containing at least one antiviral agent.

4. The pharmaceutical composition of claim 2, still additionally containing at least one interferon.

5. The pharmaceutical composition of claim 4, still additionally containing at least one interferon.

6. The pharmaceutical composition of claim 5, wherein said at least one antiviral agent is ribavirin and said at least one interferon is α-interferon or pegylated interferon.

7. A method of treating disorders associated with the HCV, said method comprising administering to a patient in need of such treatment a pharmaceutical composition which comprises therapeutically effective amounts of at least one compound of claim 1, or pharmaceutically acceptable salt thereof.

8. The method of claim 7, wherein said administration is oral or subcutaneous.

9. A pharmaceutical composition for treating disorders associated with the HCV, said composition comprising therapeutically effective amounts of at least one compound of claim 1, or pharmaceutically acceptable salt thereof, and a pharmaceutically acceptable carrier.

10. A method of treatment of a hepatitis C virus associated disorder, comprising administering to a patient in need thereof an effective amount of at least one compound of claim 1, or pharmaceutically acceptable salt thereof.

11. A method of modulating the activity of hepatitis C virus (HCV) protease, comprising contacting HCV protease with at least one compound of claim 1, or pharmaceutically acceptable salt thereof.

12. A method of treating, preventing, or ameliorating one or more symptoms of hepatitis C, comprising administering to a patient in need thereof therapeutically effective amounts of at least one compound of claim 1, or pharmaceutically acceptable salt thereof.

13. The method of claim 12, wherein the HCV protease is the NS3/NS4a protease.

14. The method of claim 13, wherein the compound or compounds inhibit HCV NS3/NS4a protease.

15. A method of modulating the processing of hepatitis C virus (HCV) polypeptide, comprising contacting a composition containing the HCV polypeptide under conditions in which said polypeptide is processed with at least one compound of claim 1.
